(19) United States
(12) Patent Application Publication
(10) Pub. No.: US 2026/0274944 A1
(43) Pub. Date: Sep. 17, 2026

(54) RAGE RECEPTOR-BINDING MOLECULES, CONJUGATES THEREOF AND THEIR USES TO DETECT, PREVENT OR TREAT LUNG DISEASES

(71) Applicants: VECT-HORUS, MARSEILLE (FR); UNIVERSITE D'AIX-MARSEILLE, MARSEILLE (FR); CENTRE NATIONAL DE LA RECHERCHE SCIENTIFIQUE, PARIS (FR)

(72) Inventors: ROMY COHEN, MARSEILLE (FR); MARION DAVID, MARSEILLE (FR); MICHEL KHRESTCHATIDKY, MARSEILLE (FR)

(21) Appl. No.: 19/123,814

(22) PCT Filed: Oct. 26, 2023
(86) PCT No.: PCT/EP2023/079982

§ 371 Date: Apr. 24, 2025

(30) Foreign Application Priority Data
Oct. 27, 2022 (EP) 22306629.1

(51) Int. Cl. C07K 16/28; A61K 47/68; A61K 49/00

(52) U.S. Cl. CPC C07K 16/2803; A61K 47/6849; A61K 49/0002; C07K 2317/24; C07K 2317/569

(57) ABSTRACT

The invention relates to Variable Domain of Camelid Heavy Chain-only (VHH) molecules that bind RAGE and the uses thereof e.g., to transport molecules of pharmaceutical or diagnostic interest into lung cells and organs, in pathological conditions including infectious, inflammatory, cancer or rare lung diseases.

The invention relates to Receptor for Advanced Glycation End products (RAGE)-binding molecules and the uses thereof. The invention more particularly relates to Variable Domain of Camelid Heavy Chain-only (VHH) molecules, which bind RAGE at the surface of cells, in particular lung epithelial cells, and uses thereof e.g., to transport molecules of pharmaceutical or diagnostic interest to lung cells and detect or treat lung diseases such as cancer, infectious, or inflammatory lung diseases.

### BACKGROUND

The treatment of lung diseases including lung cancer, lung inflammation and lung infection, such as tuberculosis, is a challenging problem in clinical practice. This is because conventional drug delivery systems cannot effectively deliver drugs to the lung via systemic route. Therefore, lung-targeted drug delivery systems that can deliver therapeutic drugs to the lung in order to increase drug concentration in lung tissue while reducing drug distribution in other organs and tissues is the ideal strategy sought by practitioners seeking in particular to treat lung diseases. Several strategies have been developed so far such as pulmonary inhalation. However, the drugs delivered via inhalation are exposed to multiple clearance mechanisms which constitute the main barriers to drug absorption following pulmonary administration (He, Gui, J. et al., 2022). Furthermore, an inhaler device is required for the drug delivery. Therefore, there is a need to develop more efficient strategies capable of enhancing the transport of therapeutic molecules specifically to the lung.

Various cellular membrane receptors targeting strategies have been developed over the past 30 years in order to optimize drug delivery. However, despite progress made in the field of drug delivery, there is still an unsatisfied need in the art for agents capable of improving drug access to the lungs.

Inventors now herein provide such advantageous ligands. Indeed, they have discovered, and herein disclose for the first time, ligands targeting a receptor expressed at high level in lung and at low level in other tissues, namely the Receptor for Advanced Glycation end products (RAGE). These ligands consist in the variable domain of heavy chain only antibodies (VHH) found in camelids. Some of the VHHs of the invention present cross-species reactivity toward the human and mouse RAGE. Inventors demonstrated and herein reveal that the identified RAGE targeting VHHs can address to the lung at high level, in a time-dependent manner, a cargo which is an antibody fragment, preferably a human IgG1 Fc.

Overexpression and activation of RAGE by its ligands such as Advanced Glycation End Products is related to inflammatory processes present in some neurodegenerative disorders, diabetic nephropathy, non-diabetic vascular disease, acute liver, lung injury and malignancies (Sims, Rowe et al. 2010). RAGE is highly expressed in the lungs under non-pathological conditions (Chavakis, Bierhaus et al. 2004, Khaket, Kang et al. 2019). To date, small molecules that act as RAGE antagonists have been developed and studied in the clinic, and therapeutic antibodies conjugated to drugs to treat certain cancers (endometrial cancer) have been evaluated in the pre-clinical phase (Healey, Pan-Castillo et al. 2019). VHH molecules have also been developed as human RAGE antagonists (Mohammed, Zeng et al. 2021). However, cross-reactive VHH-type molecules that can be used as vectors to deliver molecules of therapeutic interest to the lungs have not been developed so far. In particular, the art does not provide any efficient VHH-type molecules usable to detect (diagnose), prevent or treat lung diseases in the human being.

### SUMMARY OF THE INVENTION

The present invention provides novel binding molecules, which can be used to effectively transport molecules to the lungs. More particularly, the invention discloses VHH molecules (“VHHs”), in particular VHHs which bind both non-human and human RAGE, and which can deliver a pharmaceutical agent, typically a drug, in particular a biological agent, to the lungs. The invention demonstrates that VHH molecules of the invention can effectively accumulate in the lungs and deliver conjugated therapeutic or imaging agents in vivo in the organ, in particular into lung cells. Such VHHs thus represent very advantageous molecules for use in therapy or diagnostic.

An object of the invention thus relates to VHH molecules that bind human and non-human (e.g., rodent, such as murine or rat) RAGE.

A further object of the invention is a VHH molecule that binds a human and/or a non-human RAGE at the surface of lung cells.

Preferred VHHs of the invention bind both human and rodent, in particular murine, RAGE, can target lung cells, and have an affinity for RAGE (Kd) below 10 μM, for example of 0.1 nM to 10 μM, preferably from 1 nM to 10 μM.

A preferred object is a VHH molecule of formula FR1-CDR1-FR2-CDR2-FR3-CDR3-FR4, wherein said VHH molecule binds a Receptor for Advanced Glycation End products (RAGE) at the surface of lung cells.

In a particular aspect, the VHH molecule comprises, or consists of, one or more of the following sequences:
a CDR1 sequence selected from SEQ ID NOs: 1, 5, 9, 13 and 17,
a CDR2 sequence selected from SEQ ID NOs: 2, 6, 10, 14 and 18, and/or
a CDR3 sequence selected from SEQ ID NOs: 3, 7, 11, 15 and 19.

In another particular aspect, the VHH molecule of the invention comprises, or consists of:
a CDR1 sequence selected from SEQ ID NOs: 1, 5, 9, 13 or 17, or a variant thereof having at least 60% amino acid identity to any one of said sequences over the entire length thereof,
a CDR2 sequence selected from SEQ ID NOs: 2, 6, 10, 14 or 18, or a variant thereof having at least 60% amino acid identity to any one of said sequences over the entire length thereof, and/or
a CDR3 sequence selected from SEQ ID NOs: 3, 7, 11, 15 or 19, or a variant thereof having at least 60% amino acid identity to any one of said sequences over the entire length thereof,
said VHH having a RAGE-binding capacity at the surface of a lung cell.

In another particular aspect, the VHH molecule comprises, or consists of, SEQ ID NOs: 1, 2 and 3; or SEQ ID NOs: 5, 6 and 7; or SEQ ID NOs: 9, 10 and 11; or SEQ ID NOs: 13, 14 and 15; or SEQ ID NOs: 17, 18 and 19.

In a further particular aspect, the VHH molecule comprises, or consists of, an amino acid sequence selected from any one of SEQ ID NOs: 4, 8, 12, 16 and 20, wherein said amino acid sequence optionally comprises a tag and/or a linker.

In again another particular aspect, the VHH molecule comprises i) a tag such as a Q-tag preferably comprising or consisting of sequence LQR, a myc tag (EQKLISEEDL, SEQ ID NO: 38), a poly-His tag, a poly-Arg tag, a poly-Lys tag, an HA tag, a FLAG tag, a GFP tag, a CBP tag, a Strep II tag, a sortase-tag, a SNAP-tag or a combination thereof; and/or ii) a linker such as, for example, a Gly linker or a Ala linker.

In a further particular aspect, the VHH molecule comprises i) a tag sequence consisting of AAAEQKLISEEDLNGAAHHHHHHGS (SEQ ID NO: 36), wherein bold is an Ala linker, simple underline is a myc tag and double underline is a 6His tag; or ii) a tag sequence consisting of GGGGSCHHHHHH (SEQ ID NO: 74), wherein simple underline is a Gly linker and double underline is a 6His tag.

The invention also relates to chimeric agents (also interchangeably called herein “conjugates”) comprising one or more VHHs as defined above conjugated to at least one additional compound (/molecule). This additional compound may be a distinct VHH or a molecule which is not a VHH.

More generally, the at least one additional compound may be a stabilizing group or scaffold, which may be selected from an antibody or a fragment thereof such as a Fc fragment, a VHH molecule, PEG, a serum albumin protein or a serum albumin-binding moiety (e.g., a protein, a peptide, or a chemical molecule). In another aspect, the at least one additional compound may be a therapeutic, diagnostic or imaging compound, or a vehicle comprising such a therapeutic, diagnostic or imaging compound. In a further particular aspect, the chimeric agent/conjugate may comprise both kind of additional compounds, i.e. i) a stabilizing group or scaffold and ii) a therapeutic, diagnostic or imaging compound, or a vehicle comprising the same.

The molecule conjugated to VHH may be e.g., any active compound useful in medicine (also herein identified as a “substance of interest”) such as a “diagnostic agent” for example a tracer, or a “therapeutic agent” for example a peptide, a polypeptide, a protein, an antibody or a fragment thereof and a nucleic acid. The chimeric agent may in addition comprise a vehicle comprising the substance of interest. This vehicle may be selected for example from a virus, a virus-like particle (VLP), a Cell-Derived Vesicle (CDV), an exosome, a lipid vehicle and a polymer vehicle, and is preferably a lipid nanoparticle (LNP), a micelle or a liposome.

As indicated herein above, the chimeric agent may also contain, in addition to or instead of said active compound, a stabilizing group (e.g., a Fc, an IgG, albumin, an albumin binding molecule also called herein an albumin-binding moiety, or PEG, for instance) to increase the plasma half-life of the VHH or of the conjugate. Particular chimeric agents of the invention thus comprise i) at least one (one or more) VHH molecule, ii) a stabilizing group, iii) an active compound, typically a therapeutic, diagnostic or imaging compound, and optionally iv) a vehicle, in any order (for example a conjugate VHH-Fc-therapeutic agent).

The invention further provides pharmaceutical or diagnostic compositions comprising a chimeric agent as defined above and, optionally, a suitable (pharmaceutically acceptable) support or excipient.

The invention also provides nucleic acids, vectors, and (recombinant) host cells encoding or containing a VHH molecule or chimeric agent as defined above or as herein described.

The invention further provides methods for making a chimeric agent, comprising conjugating one or more VHH as defined above to a molecule or agent or scaffold, covalently or non-covalently.

Another object of the invention relates to a VHH molecule or chimeric agent as defined above for use to prepare a medicament or diagnostic agent, or for use as a medicament or diagnostic agent.

Another object of the invention relates to the use of a VHH molecule as defined above for increasing the biological activity and/or lung delivery of any substance of interest, typically of a diagnostic or therapeutic substance.

Another object of the invention relates to a method for improving the distribution of a molecule to the lung site, comprising coupling said molecule to a VHH as defined above.

Another object of the invention is a method for treating a pathology in a subject comprising administering to the subject a conjugate as defined above.

Another object of the invention is a method for imaging a particular cell type, target tissue or organ, typically lung cells or the lung organ, in a subject, wherein the method comprises a step of administering to the subject a conjugate as defined above.

Another object of the invention is an improved method for treating a lung pathology in a subject in need thereof with a substance of interest, typically with a therapeutic agent or drug, wherein the method comprises a step of administering to the subject a VHH, preferably a conjugate as defined above.

The invention can be used in any mammal, in particular in any human being.

### LEGENDS TO THE FIGURES

FIG. 1. RAGE expression in tissues from Pig, Rhesus Monkey, Rat and Mouse. (A) Western blots performed on total membrane fractions of the following organs: heart, muscle, kidney, cornea, retina, bladder, adrenal, pancreas, testis, stomach, lung and liver from pig, non-human primate (NHP; rhesus macaque), rat and mouse. The amount of protein loaded is indicated. (B) Immunohistochemical (IHC) detection of RAGE by an anti-RAGE antibody (R&D Systems #Mab 1179) followed by a donkey anti-rat-A488 secondary antibody (green) on cryosections (18 μm) of rat (B1) and mouse (B3) lung. Cell nuclei are labeled with Hoechst #33258 (blue). No green labelling appears on rat (B2) and mouse (B4) lung when IHC is performed with the secondary antibody only.

FIG. 2. Validation of CHO cell lines expressing the human or mouse RAGE. (A) Map of the plasmid construct used to generate the various h/mRAGE-GFP expressing cell lines. (B) Validation of receptor expression in CHO cell line overexpressing the human form of RAGE by immunocytochemical experiments. Cell nuclei were labeled with Hoechst #33258 (blue). RAGE was detected with an anti-RAGE antibody, followed by Alexa594-conjugated secondary antibodies (red). Co-labeling of RAGE-EGFP (green) and anti-RAGE antibody appears in yellow in the merged picture. (C) Validation of receptor expression in CHO cell lines by western blot experiments on cell membrane preparations, using a RAGE specific antibody, followed by HRP-conjugated secondary antibody.

FIG. 3. Apparent K_{d} determination of VHHs on hRAGE- and mRAGE-expressing CHO cell lines. (A) CHO-hRAGE-EGFP and CHO-mRAGE-EGFP cells were incubated 1 hr at 4° C. with increasing concentrations of VHHs, detected with a mouse anti-6His (1/1000) and an Alexa647-conjugated anti-mouse secondary antibody (1/400). Measurements were performed using flow cytometry. The ratio of fluorescence intensity for each point was normalized with the corresponding EGFP signal (receptor expression) and gave rise to the arbitrary units. Data are presented as mean±SEM of at least 3 independent experiments. (B) Characteristics of selected VHHs: Molecular Weight (Da); Apparent K_{d} on human RAGE (nM); Apparent K_{d} on mouse RAGE (nM). Data are presented as mean±SEM of at least 3 independent experiments.

FIG. 4. Apparent K_{d} determination of VHH-siRNAs on a mRAGE-expressing CHO cell line. (A) CHO-mRAGE-EGFP cells were incubated 1 hr at 4° C. with increasing concentrations of VHH-siRNAs, detected with a mouse anti-6His (1/1000) and an Alexa647-conjugated anti-mouse secondary antibody (1/400). Measurements were performed using flow cytometry. (B) Characteristics of selected VHH-siRNAs: Molecular Weight (Da); Apparent K_{d} on mouse RAGE (nM).

FIG. 5. Cellular binding/uptake of VHH-Fcs on CHO cells expressing mRAGE. Representative confocal photomicrographs of CHO-mRAGE-EGFP cells (green) incubated 1 hr at 37° C. with VHH 1-Fc, VHH 2-Fc, VHH 3-Fc, VHH 4-Fc, VHH 5-Fc and with the control VHH ctrl-Fc at 50 nM, detected using an Alexa594-conjugated anti-hFc antibody (1/800) post-PFA fixation and following X-100 permeabilization of cell membranes (red). Cell nuclei were labeled with Hoechst #33342 at 0.5 μg/ml (blue). Co-labeling appears in yellow/orange in the merged pictures.

FIG. 6. Cellular binding/uptake of VHH-Fcs on CHO cells expressing hRAGE. Representative confocal photomicrographs of CHO-hRAGE-EGFP cells (green) incubated 1 hr at 37° C. with VHH 1-Fc, VHH 2-Fc, VHH 3-Fc, VHH 4-Fc, VHH 5-Fc and with the control VHH ctrl-Fc at 50 nM, detected using an Alexa594-conjugated anti-hFc antibody (1/800) post-PFA fixation and following X-100 permeabilization of cell membranes (red). Cell nuclei were labeled with Hoechst #33342 at 0.5 μg/ml (blue). Co-labeling appears in yellow/orange in the merged pictures.

FIG. 7. Apparent K_{d} determination of VHH-Fcs and Fc-VHHs on hRAGE- and mRAGE-expressing CHO cell lines. (A) CHO-hRAGE-EGFP and CHO-mRAGE-EGFP cells were incubated 1 hr at 4° C. with increasing concentrations of VHH-Fcs or Fc-VHHs, detected with an Alexa647-conjugated anti-hFc antibody (1/400). Measurements were performed using flow cytometry. The ratio of fluorescence intensity for each point was normalized with the corresponding EGFP signal (receptor expression) and gave rise to the arbitrary units. Data are presented as mean±SEM of at least 3 independent experiments. (B) Characteristics of selected VHH-Fcs and Fc-VHHs: Molecular Weight (Da); Apparent K_{d} on human RAGE (nM); Apparent K_{d} on mouse RAGE (nM). Data are presented as mean±SEM of at least 3 independent experiments.

FIG. 8. Competition assay between VHHs and VHH 1-Fc. (A) Principle of the competition test. In a first step, CHO-mRAGE-EGFP cells were incubated 1 hr at 4° C. with the competitor in dilution series. Second, the tracer at EC80-90 was added and incubated for 1 hr at 4° C. Tracer was then revealed with the appropriate revelation system. Measurements were performed using flow cytometry. (B) CHO-mRAGE-EGFP cells were incubated with the competitors (VHHs). Tracer (VHH 1-Fc) at EC80 was then added and detected with Alexa647-conjugated anti-hFc antibody (1/400).

FIG. 9. Identification of the mRAGE domains that interact with VHH-Fcs. (A) Schematic representation of full length and truncated constructs of mRAGE. The mRAGE extracellular moiety has been truncated sequentially of its domains generating 2 truncated variants. A HA tag was added to verify appropriate extracellular localization of the extracellular domains. (B) Representative confocal photomicrographs of CHO cells transiently expressing mRAGE, HA-mRAGE, mRAGE−ΔV (=ΔV) and mRAGE−ΔV−C1 (=ΔV−C1) (all in green), incubated 1 hr at 37° C. with 250 nM VHH 5-Fc, detected post-PFA fixation with an Alexa594-conjugated anti-hFc antibody (1/800, red). Cell nuclei were labeled with Hoechst #33342 at 0.5 μg/ml (blue). Co-labeling appears in yellow/orange in the merged pictures. (C) Table summarizing binding properties between the anti-HA antibody or VHH-Fcs with full length and truncated mRAGE transiently expressed in CHO cell determined by immunocytochemistry experiments. (+) means positive binding. (−) means no binding.

FIG. 10. Cellular binding/co-localization of VHH-Fc on RAGE in mice lungs. Immunocytochemistry was performed on the lungs of mice injected with VHH ctrl-Fc, VHH 1-Fc and VHH 5-Fc into tail vein at 35 nmol/kg. The mice were perfused with saline 48 hours post injection and the lungs were incubated in PFA 4% overnight. Lungs were extensively washed in PBS 1× and incubated 2 days in sucrose 30%. Double-immunofluorescent staining of lung tissue with anti-RAGE (Alexa Fluor 488, green) and with VHH-Fc (Alexa Fluor 594, red) antibodies were performed on lung sections. Cell nuclei were labeled with Hoechst #33342 at 0.5 μg/ml (blue). Co-labeling appears in yellow/orange in the merged pictures (right panel). Representative photographs were taken with a confocal microscope with 20× and 63× magnification.

FIG. 11. A. Distribution and lung uptake of VHH-Fc fusion in WT C57Bl/6 mice. VHH ctrl-Fc, VHH 1-Fc, VHH 4-Fc and VHH 5-Fc were injected into tail vein at 35 nmol/kg and the mice were perfused with saline at 2, 6, 18, 48, 96 or 168 hours post injection. Amounts of VHH-Fc in plasma and lung were assessed by ELISA. VHH-Fc concentration in plasma (A, C) and percentage of injected dose per gram of tissue in plasma (B, D). VHH-Fc concentration in lung (E), percentage of injected dose per gram of tissue in lung (F) and lung-to-plasma ratio (G). Data represent the mean±SD. N=4-12 per group per time point; *p≤0.05 for VHH 1-Fc, 4 or 5 vs VHH ctrl-Fc; **p≤0.01; ***p≤0.001. B. Distribution and liver and kidney uptake of VHH-Fc fusion in WT C57Bl/6 mice. VHH ctrl-Fc, VHH 1-Fc, VHH 4-Fc and VHH 5-Fc were injected into tail vein at 35 nmol/kg and the mice were perfused with saline at 6, 18, 48, 96 or 168 hours post injection. Amounts of VHH-Fc in liver and kidney were assessed by ELISA. VHH-Fc concentration in liver (H), percentage of injected dose per gram tissue in liver (I) and liver-to-plasma ratio (J). VHH-Fc concentration in kidney (K), percentage of injected dose per gram tissue in kidney (L) and kidney-to-plasma ratio (M). Data represent mean±SD. N=4-12 per group per time point; *p≤0.05 for VHH 1-Fc, 4 or 5 vs VHH ctrl-Fc; **p≤0.01; ***p≤0.001.

FIG. 12. VHH conjugation strategies. Using either chemical conjugation or recombinant fusion, VHHs can be used to vectorize numerous types of molecules, including for example and non-exhaustively imaging and radiotherapeutic agents as well as small organic molecules, dyes, peptides, proteins including antibodies, nucleic acids including siRNAs and antisens oligonucleic acids (ASOs), nanoparticles (NPs), or liposomes. Moreover, VHHs can be used to vectorize a molecule while being in the form of a monovalent (VHH) or multivalent (VHHₙ) conjugate.

FIG. 13. VHH-RAGE LNP characterization, in vitro mLuc mRNA delivery to h/mRAGE-GFP CHO cells and in vivo distribution in C57/B16 mice at 6 hr. (A) LNPs were functionalized with either VHH ctrl or VHH5, loaded with mLuc mRNA, and characterized by DLS analysis to determine their Z-average size and PDI (Polydispersity Index). Data represent the mean of N=3 measurements. Additionally, LNPs were characterized for lipid concentration using a standard cholesterol assay and for mRNA concentration using a RiboGreen assay to estimate the N/P ratio. (B) CHO-hRAGE-EGFP or CHO-mRAGE-EGFP cells (20000 cells/well of a 96-well plate) were incubated 6 hours at 37° C. with naked LNP, VHH ctrl-LNP and VHH5-LNP at a concentration equivalent of 1.25 μg of mLuc mRNA/mL in OptiMEM culture medium. The capacity of naked or functionalized LNPs to deliver mLuc mRNA inside cells was assessed by quantifying the luminescence produced by the translated luciferase protein using the ONE-Glo™ Luciferase Assay System (Promega). Luminescence was measured using a GloMax navigator (Promega). Data represent the mean of Relative Light Unit (RLU)±SEM. N=3 per group per condition; ***p≤0.001 for VHH 5-LNP, vs VHH ctrl. (C) VHH ctrl-LNP and VHH 5-LNP were injected into tail vein of C57/B16 mice at 20 μg/200 μL of mLuc mRNA equivalent per mice and the organs were collected 6 hours post injection. Lung, muscle (gastrocnemius), heart, kidney and brain were crushed before luminescence was quantified using the ONE-Glo™ Luciferase Assay System. Data represent the mean of Relative Light Unit (RLU)±SEM. N=3/4 mice per LNP formulation *p≤0.05 for VHH 5-LNP, vs VHH ctrl; **p≤0.01.

### DETAILED DESCRIPTION OF THE INVENTION

The present invention provides novel RAGE-binding agents which can be used to transport molecules, such as therapeutic, imaging or diagnostic agents, to the lungs. More particularly, the invention discloses improved VHH molecules which bind RAGE, and uses thereof.

The Receptor for Advanced Glycated Endproducts (RAGE) is a 45 kDa transmembrane receptor member of the immunoglobulin super family. Structurally, full-length human RAGE consists of three major domains:
a V-type (variable) domain followed by two C-type (constant) domains usually termed C1 and C2;
a single hydrophobic transmembrane domain; and
a short charged intracellular cytoplasmic domain, which is primarily associated with signaling (Neeper et al., J. Biol. Chem. 1992).

In addition to the full-length, membrane-bound forms, there are soluble forms of RAGE. Soluble RAGE contains only the N-terminal and extracellular domains and is a product of either alternative splicing or of proteolysis of RAGE by ADAM10 or matrix metalloproteinases (Raucci A et al. FASEB J. 2008, Yonekura H et al. Biochem J. 2003, Hudson B et al. FASEB J. 2008).

In human adult, RAGE is expressed in most tissues but at very low levels except in the lung (Brett J et al. Am J Pathol. 1993). RAGE recognizes a variety of ligands such as Advanced Glycation End Products (AGE), high-mobility group box protein (HMGB1), macrophage-antigen-1 (Mac1), S-100 proteins, β-amyloid peptide, DNAs, etc. Most of them bind RAGE to the V-domain. S100A12, Aβ and S100A6 have been reported to bind RAGE on the C1/C2 domain (Lee and Park. Genomics Inform. 2013, Ostendorp et al. EMBO J. 2007, Leclerc E et al, J Biol Chem. 2007).

Ligand binding to RAGE initiate multiple cellular cascade responses leading to inflammation. Activation of the transcription factors NF-κB, MAP kinases, ERK1 and ERK2 or p21ras result in the transcription of several target genes including cytokines (IL-1β, IL-6, TNF-α), adhesion molecules and RAGE itself (Bongarzone S et al. J Med Chem. 2017). This positive feedback loop between RAGE and NF-κB, by which the RAGE signal is maintained and amplified, contributes to chronic, pathological inflammation in many diseases (Bierhaus A et al. Diabetes. 2001, Sparvero J et al. J Trans Med. 2009, Dong H et al. Front Immunol. 2022). The diversity of signaling cascades suggests that various RAGE ligands may activate different signaling pathways in different cell types.

Inventors now advantageously herein reveal that conjugating drugs or diagnostic agents to a VHH that targets RAGE is advantageous to deliver such a molecule of interest, for example a diagnostic agent or a drug, to tissues that preferentially express RAGE, in particular the lung, for the diagnostic or treatment of a disease.

Using purified membrane preparations from cells expressing high levels of hRAGE and mRAGE, inventors generated and selected VHH molecules, in particular VHH molecules that bind both the human and non-human RAGE. They showed that when fused to a human IgG1 Fc region or a siRNA, these VHH molecules retain RAGE binding capacity. They also showed that VHH molecules display efficient lung delivery. They finally demonstrated that RAGE-targeting VHHs molecules of the invention can also be used to vectorize lipid nanoparticle (LNP), and that such conjugates can be used to effectively transport and deliver to the lungs various molecules of diagnostic or therapeutic interest such as mRNA/LNP.

The invention thus provides novel RAGE-binding molecules which represent valuable agents for drug targeting to the lung.

An object of the invention thus relates to VHH molecules, wherein said VHH molecules bind both a human and a non-human (e.g., rodent, such as rat or murine) RAGE. Preferably, the VHH binds RAGE-expressing lung tissues. The invention also relates to chimeric agents comprising such VHH, their manufacture, compositions comprising the same and the use thereof.

### VHH Molecules

VHH molecules correspond to the variable region of heavy chain only camelid antibodies that are naturally devoid of light chains. VHH have a very small size of around 15 kDa. They contain a single chain molecule that can bind its cognate antigen using a single domain. The antigen-binding surfaces of VHHs are usually more convex (or protruding) than those of conventional antibodies, which are usually flat or concave. More specifically, VHHs are composed of 4 Framework Regions (or FRs) whose sequences and structures are defined as conserved, and three Complementarity Determining Regions (or CDRs) showing high variability both in sequence content and structure conformation, which are involved in antigen binding and provide antigen specificity. Compared to conventional human antibody VH, a few amino acids are substituted in the FR2 region and complementarity-determining regions (CDRs) of VHH. For instance, highly conserved hydrophobic amino acids (such as Val42, Gly49, Leu50, and/or Trp52) in FR2 region are often replaced by hydrophilic amino acids (Phe42, Glu49, Arg50, Gly52), rendering the overall structure more hydrophilic and contributing to high stability, solubility and resistance to aggregation.

VHH molecules according to the present invention are polypeptides comprising (or consisting of, or consisting essentially of) an antigen-binding domain of a heavy chain only antibody (HcAb).

In order to generate VHH molecules having suitable properties, the inventors tested over 700 RAGE-binding VHH from a library of VHH produced by lama immunization with a RAGE immunogen. Following analysis of said clones for binding and specificity, the inventors further selected about 70 clones which had the required specificity and cross species binding. Said clones were all sequenced and their structure was analyzed and compared. The sequences of the relevant domains and preferred VHH are provided in the experimental section and sequence listing. The properties of the VHH and conjugates thereof are also illustrated in the experimental section.

VHH molecules of the invention typically comprise or consist of the formula:

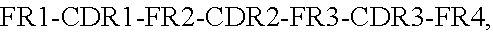

wherein FRn designates framework regions and CDRn designates complementarity determining regions [and wherein n is for example 1, 2, 3 or 4].

In a particular embodiment, VHH molecules of the invention comprise a CDR1 domain comprising or consisting of an amino acid sequence selected from SEQ ID NOs: 1, 5, 9, 13 or 17, or variants thereof having at least 60%, in particular at least 65%, 70% or 75%, for example at least 80% or 85% amino acid identity to any one of said sequences over the entire length thereof, preferably at least 85%, 86%, 87%, 88%, 89%, 90%, 91%, 92%, 93%, 94%, 95%, 96%, 97%, 98% or 99% (the preferred percentages of identity for a particular sequence being preferably percentages corresponding to an integer number of amino acids), said variants retaining a RAGE binding capacity. Preferred VHH molecules of the invention contain a CDR1 domain having an amino acid sequence selected from SEQ ID NOs: 1, 5, 9, 13 or 17, or variants thereof having several amino acid modifications, for example at least 3 amino acid modifications, preferably at most 3 or 2 amino acid modifications, in a particular aspect at most 1 amino acid modification.

The “% identity” between amino acid (or nucleic acid) sequences may be determined by techniques known per se in the art. Typically, the % identity between two nucleic acid or amino acid sequences is determined by means of computer programs such as GAP provided in the GCG program package (Program Manual for the Wisconsin Package, Version 8, August 1996, Genetics Computer Group, 575 Science Drive, Madison, Wisconsin, USA 53711) (Needleman, S. B. and Wunsch, C. D., (1970), Journal of Molecular Biology, 48, 443-453). The % identity between two sequences designates the identity over the entire length of said sequences. As indicated above, the preferred percentages of identity for a particular sequence are preferably percentages corresponding to an integer number of amino acids both in the reference sequence (which is for example SEQ ID NOs: 1, 5, 9, 13 or 17 or any other reference sequences herein identified such as SEQ ID NOs: 2, 3, 6, 7, 10, 11, 14, 15, 18 or 19) and in the variant thereof.

Specific examples of VHH molecules of the invention comprise a CDR1 sequence comprising, or consisting essentially of, SEQ ID NOs: 1, 5, 9, 13 or 17.

In a further particular embodiment, VHH molecules of the invention comprise a CDR2 domain comprising or consisting of an amino acid sequence selected from SEQ ID NOs: 2, 6, 10, 14 or 18, or variants thereof having at least 60%, in particular at least 65%, 70% or 75%, for example at least 80% or 85%, amino acid identity to any one of said sequences over the entire length thereof, preferably at least 85%, 86%, 87%, 88%, 89%, 90%, 91%, 92%, 93%, 94%, 95%, 96%, 97%, 98% or 99%, said variants retaining a RAGE binding capacity. Preferred VHH molecules of the invention contain a CDR2 domain having an amino acid sequence selected from SEQ ID NOs: 2, 6, 10, 14 or 18, or variants thereof having several amino acid modifications, for example at least 3 amino acid modifications, preferably at most 3 or 2 amino acid modifications, in a particular aspect at most 1 amino acid modification.

Specific examples of VHH molecules of the invention comprise a CDR2 sequence comprising, or consisting essentially of, SEQ ID NOs: 2, 6, 10, 14 or 18.

In a further particular embodiment, VHH molecules of the invention comprise a CDR3 domain comprising or consisting of an amino acid sequence selected from SEQ ID NOs: 3, 7, 11, 15 or 19, or variants thereof having at least 60%, in particular at least 65%, 70% or 75%, for example at least 80% or 85%, amino acid identity to any one of said sequences over the entire length thereof, preferably at least 85%, 86%, 87%, 88%, 89%, 90%, 91%, 92%, 93%, 94%, 95%, 96%, 97%, 98% or 99%, said variants retaining a RAGE binding capacity. Preferred VHH molecules of the invention contain a CDR3 domain having an amino acid sequence selected from SEQ ID NOs: 3, 7, 11, 15 or 19, or variants thereof having several amino acid modifications, for example at least 3 amino acid modifications, preferably at most 3 or 2 amino acid modifications, in a particular aspect at most 1 amino acid modification.

Specific examples of VHH molecules of the invention comprise a CDR3 sequence comprising, or consisting essentially of, SEQ ID NOs: 3, 7, 11, 15 or 19.

In a further particular embodiment, VHH molecules of the invention comprise:
a CDR1 domain comprising, or consisting of, an amino acid sequence selected from SEQ ID NOs: 1, 5, 9, 13 or 17, or variants thereof having at least 60%, in particular at least 65%, 70% or 75%, for example at least 80% or 85%, amino acid identity to any one of said sequences over the entire length thereof, preferably at least 85%, 86%, 87%, 88%, 89%, 90%, 91%, 92%, 93%, 94%, 95%, 96%, 97%, 98% or 99%, more preferably at least 95%; and
a CDR2 domain comprising or consisting of an amino acid sequence selected from SEQ ID NOs: 2, 6, 10, 14 or 18, or variants thereof having at least 60%, in particular at least 65%, 70% or 75%, for example at least 80% or 85%, amino acid identity to any one of said sequences over the entire length thereof, preferably at least 85%, 86%, 87%, 88%, 89%, 90%, 91%, 92%, 93%, 94%, 95%, 96%, 97%, 98% or 99% more preferably at least 95%; and
a CDR3 domain comprising or consisting of an amino acid sequence selected from SEQ ID NOs: 3, 7, 11, 15 or 19, or variants thereof having at least 60%, in particular at least 65%, 70% or 75%, for example at least 80% or 85%, amino acid identity to any one of said sequences over the entire length thereof, preferably at least 85%, 86%, 87%, 88%, 89%, 90%, 91%, 92%, 93%, 94%, 95%, 96%, 97%, 98% or 99%, more preferably at least 95%,
said VHH having a RAGE-binding capacity.

In a further particular embodiment, the VHH molecules of the invention comprise:
a CDR1 sequence selected from SEQ ID NOs: 1, 5, 9, 13 or 17, or a variant thereof having at least 60% amino acid identity to any one of said sequences over the entire length thereof,
a CDR2 sequence selected from SEQ ID NOs: 2, 6, 10, 14 or 18, or a variant thereof having at least 60% amino acid identity to any one of said sequences over the entire length thereof, and/or
a CDR3 sequence selected from SEQ ID NOs: 3, 7, 11, 15 or 19, or a variant thereof having at least 60% amino acid identity to any one of said sequences over the entire length thereof,
said VHH having a RAGE-binding capacity at the surface of a lung cell.

In a further particular embodiment, the VHH molecules of the invention comprise:
a CDR1 sequence selected from SEQ ID NOs: 1, 5, 9, 13 or 17, or a variant thereof having at least 60% amino acid identity to any one of said sequences over the entire length thereof,
a CDR2 sequence selected from SEQ ID NOs: 2, 6, 10, 14 or 18, or a variant thereof having at least 60% amino acid identity to any one of said sequences over the entire length thereof, and/or
a CDR3 sequence selected from SEQ ID NOs: 3, 7, 11, 15 or 19, or a variant thereof having at least 60% amino acid identity to any one of said sequences over the entire length thereof,
said VHH binding RAGE at the surface of a lung cell, with an affinity (Kd) of 0.1 nM to 10 μM, preferably from 1 nM to 10 μM.

In a preferred embodiment, the VHH molecules of the invention comprise:
a CDR1 domain having an amino acid sequence selected from the group consisting of SEQ ID NOs: 1, 5, 9, 13, 17, and variants thereof having at most 3, 2 or 1 amino acid modifications; and
a CDR2 domain having an amino acid sequence selected from the group consisting of SEQ ID NOs: 2, 6, 10, 14, 18, and variants thereof having at most 3, 2 or 1 amino acid modifications; and
a CDR3 domain having an amino acid sequence selected from the group consisting of SEQ ID NOs: 3, 7, 11, 15, 19, and variants thereof having at most 3, 2 or 1 amino acid modifications.

In a more preferred embodiment, the VHH molecules of the invention comprise a CDR1, a CDR2 and a CDR3, wherein said CDR1, CDR2 and CDR3 domains comprise or consist of, respectively:
SEQ ID NOs: 1, 2 and 3; or.
SEQ ID NOs: 5, 6 or 7; or.
SEQ ID NOs: 9, 10 and 11; or.
SEQ ID NOs: 13, 14 and 15; or.
SEQ ID NOs: 17, 18 or 19; or
variants thereof as defined above, preferably variants having at most 3, 2 or 1 amino acid modifications.

Preferred VHH molecules of the invention comprise FRs domains are defined below.

In a particular embodiment, the FR1 domain comprises or consists of SEQ ID NO: 75 as represented below, or variants thereof having at least 85%, for example at least 86%, 87%, 88%, 89%, 90%, 91%, 92%, 93%, 94%, 95%, 96%, 97%, 98% or 99%, amino acid identity to this sequence over the entire length thereof, preferably at least 90%, more preferably at least 95%:

(SEQ ID NO: 75)
EVQLVESGGGLVQPGGSLRLSCAAS.

More preferably, the bold amino acid residues are present and the variability occurs only on the other positions.

In a specific embodiment, the E in position 1 may be replaced with Q.

In a specific embodiment, the V in position 5 may be replaced with Q.

In a specific embodiment, the E in position 6 may be replaced with Q.

In a specific embodiment, the G in position 10 may be replaced with K or A.

In a specific embodiment, the L in position 11 may be replaced with V or E.

In a specific embodiment, the P in position 14 may be replaced with A.

In a specific embodiment, the A in position 23 may be replaced with V or T.

In a specific embodiment, the A in position 24 may be replaced with V.

More preferably, the FR1 contains at most 4 amino acid modifications by reference to this sequence, even more preferably at most 3, even more preferably at most 2 amino acid modifications in non-bold amino acid residues.

In a further specific embodiment, the FR1 has an amino acid sequence selected from any one of the amino acid sequences listed below:

(SEQ ID NO: 76)
QVQLVQSGGGLVQPGGSLRLSCAVS;
(SEQ ID NO: 77)
QVQLVQSGGGLVQAGGSLRLSCAAS;
(SEQ ID NO: 78)
QVQLVQSGGGLVQAGGSLRLSCVAS;
(SEQ ID NO: 79)
EVQLVESGGGLVQAGGSLRLSCVAS;
(SEQ ID NO: 80)
EVQLVESGGGLVQPGGSLRLSCAAS,

or variants thereof as defined above.

Examples of such variants are provided below, for illustration only:

(SEQ ID NO: 81)
QVQLVESGGGLVQPGGSLRLSCAAS,
(SEQ ID NO: 82)
EVQLQESGGGLVQPGGSLRLSCAAS,
(SEQ ID NO: 83)
EVQLVQSGGGLVQPGGSLRLSCAAS,
(SEQ ID NO: 84)
EVQLVESGGGLVQAGGSLRLSCAAS,
(SEQ ID NO: 85)
EVQLVESGGGLVQPGGSLRLSCVAS,
(SEQ ID NO: 86)
EVQLVESGGGLVQPGGSLRLSCAVS.

Combinations of any of such mutations may be present in other variants of interest.

In a particular embodiment, VHH molecules of the invention comprise a FR2 domain comprising or consisting of SEQ ID NO: 87 as represented below, or variants thereof having at least 85%, for example at least 86%, 87%, 88%, 89%, 90%, 91%, 92%, 93%, 94%, 95%, 96%, 97%, 98% or 99%, amino acid identity to this sequence over the entire length thereof, preferably at least 90%, or at least 95%:

(SEQ ID NO: 87)
MGWYRQAPGKQRELVAR.

More preferably, the bold amino acid residues are present and the variability occurs only on the other positions.

In a specific embodiment, the M in position 1 may be replaced with I or V.

In a specific embodiment, the G in position 2 may be replaced with A.

In a specific embodiment, the Y in position 4 may be replaced with F.

In a specific embodiment, the Q in position 6 may be replaced with R.

In a specific embodiment, the A in position 7 may be replaced with R.

In a specific embodiment, the K in position 10 may be replaced with E.

In a specific embodiment, the Q in position 11 may be replaced with E.

In a specific embodiment, the R in position 12 may be replaced with L.

In a specific embodiment, the L in position 14 may be replaced with F or W.

In a specific embodiment, the V in position 15 may be replaced with A.

In a specific embodiment, the A in position 16 may be replaced with T.

In a specific embodiment, the R in position 17 may be replaced with T or L.

More preferably, the FR2 contains at most 6 amino acid modifications by reference to this sequence, even more preferably at most 5, at most 3, even more preferably at most 2 amino acid modifications in non-bold amino acid residues.

VHH molecules as herein described typically comprise at least one of the following amino acids in the FR2 domain: Phe42, Glu49 or Arg50 (according to IMGT numbering).

In a further specific embodiment, the FR2 has an amino acid sequence selected from any one of the amino acid sequences listed below:

(SEQ ID NO: 88)
MGWYRQAPGKQRELAAR;
(SEQ ID NO: 89)
MGWYRQAPGKQREWVTT;
(SEQ ID NO: 90)
MAWFRQAPGEEREFVAR;
(SEQ ID NO: 91)
MGWYRQAPGKQLELVAL

or variants thereof as defined above.

Examples of such variants are provided below, for illustration only:

(SEQ ID NO: 92)
MAWYRQAPGKQRELVAR,
(SEQ ID NO: 93)
MGWFRQAPGKQRELVAR,
(SEQ ID NO: 94)
MGWYRQAPGEQRELVAR,
(SEQ ID NO: 95)
MGWYRQAPGKERELVAR,
(SEQ ID NO: 96)
MGWYRQAPGKQLELVAR,
(SEQ ID NO: 97)
MGWYRQAPGKQREFVAR,
(SEQ ID NO: 98)
MGWYRQAPGKQREWVAR,
(SEQ ID NO: 99)
MGWYRQAPGKQRELVTR,
(SEQ ID NO: 100)
MGWYRQAPGKQRELVAT,
(SEQ ID NO: 101)
MGWYRQAPGKQRELVAL.

Combinations of any of such mutations may be present in other variants of interest.

In a particular embodiment, VHH molecules of the invention comprise a FR3 domain comprising or consisting of SEQ ID NO: 102 as represented below, or variants thereof having at least 85% amino acid identity to this sequence over the entire length thereof, preferably at least 90%, more preferably at least 95%:

(SEQ ID NO: 102)
NYADSVKGRFTISRDNAKNTVYLQMNSLKPEDTAVYYC.

More preferably, the bold amino acid residues are present and the variability occurs only on the other positions.

In a specific embodiment, the N in position 1 may be replaced with S or D.

In a specific embodiment, the Y in position 2 may be replaced with A.

In a specific embodiment, the A in position 3 may be replaced with L.

In a specific embodiment, the D in position 4 may be replaced with A.

In a specific embodiment, the S in position 5 may be replaced with F.

In a specific embodiment, the K in position 7 may be replaced with R.

In a specific embodiment, the N in position 16 may be replaced with T.

In a specific embodiment, the A in position 17 may be replaced with T.

In a specific embodiment, the N in position 19 may be replaced with K.

In a specific embodiment, the T in position 20 may be replaced with A.

In a specific embodiment, the V in position 21 may be replaced with L.

In a specific embodiment, the N in position 26 may be replaced with I.

In a specific embodiment, the S in position 27 may be replaced with N.

In a specific embodiment, the K in position 29 may be replaced with E.

In a specific embodiment, the P in position 30 may be replaced with L.

In a specific embodiment, the V in position 35 may be replaced with R.

More preferably, the FR3 contains at most 7 amino acid modifications by reference to this sequence, even more preferably at most 6, at most 3, even more preferably at most 2 amino acid modifications in non-bold amino acid residues.

In a further specific embodiment, the FR3 has an amino acid sequence selected from any one of the amino acid sequences listed below:

(SEQ ID NO: 103)
NYLDSVKGRFTISRDNAKNTVYLQMNSLKLEDTAVYYC;
(SEQ ID NO: 104)
DYAASVKGRFTISRDTAKNAVYLQMNNLKPEDTARYYC;
(SEQ ID NO: 105)
SYADSVKGRFTISRDNAKNTVYLQMISLKPEDTAVYYC;
(SEQ ID NO: 106)
NYADFVRGRFTISRDTTKKTLYLQMNSLEPEDTAVYYC,

or variants thereof as defined above.

Examples of such variants are provided below, for illustration only:

(SEQ ID NO: 107)
SYADSVKGRFTISRDNAKNTVYLQMNSLKPEDTAVYYC
(SEQ ID NO: 108)
DYADSVKGRFTISRDNAKNTVYLQMNSLKPEDTAVYYC
(SEQ ID NO: 109)
NYLDSVKGRFTISRDNAKNTVYLQMNSLKPEDTAVYYC
(SEQ ID NO: 110)
NYAASVKGRFTISRDNAKNTVYLQMNSLKPEDTAVYYC
(SEQ ID NO: 111)
NYADFVKGRFTISRDNAKNTVYLQMNSLKPEDTAVYYC
(SEQ ID NO: 112)
NYADSVRGRFTISRDNAKNTVYLQMNSLKPEDTAVYYC
(SEQ ID NO: 113)
NYADSVKGRFTISRDTAKNTVYLQMNSLKPEDTAVYYC
(SEQ ID NO: 114)
NYADSVKGRFTISRDNTKNTVYLQMNSLKPEDTAVYYC
(SEQ ID NO: 115)
NYADSVKGRFTISRDNAKKTVYLQMNSLKPEDTAVYYC
(SEQ ID NO: 116)
NYADSVKGRFTISRDNAKNAVYLQMNSLKPEDTAVYYC
(SEQ ID NO: 117)
NYADSVKGRFTISRDNAKNTLYLQMNSLKPEDTAVYYC
(SEQ ID NO: 118)
NYADSVKGRFTISRDNAKNTVYLQMISLKPEDTAVYYC
(SEQ ID NO: 119)
NYADSVKGRFTISRDNAKNTVYLQMNNLKPEDTAVYYC
(SEQ ID NO: 120)
NYADSVKGRFTISRDNAKNTVYLQMNSLEPEDTAVYYC
(SEQ ID NO: 121)
NYADSVKGRFTISRDNAKNTVYLQMNSLKLEDTAVYYC
(SEQ ID NO: 122)
NYADSVKGRFTISRDNAKNTVYLQMNSLKPEDTARYYC.

Combinations of any of such mutations may be present in other variants of interest.

In a particular embodiment, VHH molecules of the invention comprise a FR4 domain comprising or consisting of SEQ ID NO: 123 as represented below, or variants thereof having at least 85%, for example at least 86%, 87%, 88%, 89%, 90%, 91%, 92%, 93%, 94%, 95%, 96%, 97%, 98% or 99%, amino acid identity to this sequence over the entire length thereof, preferably at least 90%, more preferably at least 95%:

(SEQ ID NO: 123)
WGQGTQVTVSS.

More preferably, the bold amino acid residues are present and the variability occurs only on the other positions.

More preferably, the FR4 contains at most 4 amino acid modifications by reference to this sequence, even more preferably at most 3, even more preferably at most 2 amino acid modifications in non-bold amino acid residues.

A specific illustrative example of a FR4 sequence is WGKGTQVTVSS (SEQ ID NO: 124) or WGQGTQVTVSS (SEQ ID NO: 125).

Specific examples of RAGE-binding VHH molecules of the invention are molecules comprising or consisting of an amino acid sequence selected for example from any one of SEQ ID NOs: 4 (VHH 1, x=0), 8 (VHH 2, x=0), 12 (VHH 3, x=0), 16 (VHH 4, x=0), 20 (VHH 5, x=0), 31 (VHH 1, x=1), 32 (VHH 2, x=1), 33 (VHH 3, x=1), 34 (VHH 4, x=1), and 35 (VHH 5, x=1), wherein each of these sequences either comprises a tag sequence (when x is 1), or not (when x is 0).

In the herein above provided examples corresponding to SEQ ID NO: 31, 32, 33, 34 and 35 wherein x is equal to 1, each VHH molecule comprises the following particular tag sequence of SEQ ID NO: 36: AAAEQKLISEEDLNGAAHHHHHHGS. If x is equal to 0 such as in the examples corresponding to SEQ ID NO: 4, 8, 12, 16 and 20, the VHH molecule does not comprise any tag.

In a particular embodiment, the VHH molecule of the invention is humanized.

For humanization, one or more of the FR domains may be (further) modified by one or more amino acid substitutions.

In this respect, in a particular embodiment, the VHHs are humanized by selected modifications (e.g., amino acid substitution) of the FR1 domain. A FR1 domain consists typically in a sequence of 25 amino acid residues. A typical humanized position in FR1 is selected from 14P and 23A (for example by reference to any one of SEQ ID NOs: 75, 76, 77 or 80, or to any variants thereof as herein defined, in particular as herein exemplified such as any one of SEQ ID NOs: 81-86). A particular example of a humanized FR1 comprises SEQ ID NO: 83. A14 and/or V23 which are present in SEQ ID NO: 78 are in particular respectively modified into 14P or 23A in the humanized sequence of SEQ ID NO: 83.

In another particular embodiment, the VHH are humanized by selected modifications of the FR2 domain. A typical humanized position in FR2 is selected from 11G and 12L by reference to a FR2 domain consisting typically in a sequence of 17 amino acid residues, such as for example MGWYRQAPGKGLELVAR (SEQ ID NO: 126).

In another particular embodiment, the VHH are humanized by selected modifications of the FR3 domain. Typical humanized positions in FR3 may be selected from 17S, 21L, 27S, 29R, 30A, 35V, and any combinations thereof by reference to a FR3 domain consisting typically in a sequence of 38, at most 39, amino acid residues.

A humanized FR3 domain thus comprises a FR3 sequence of 38, at most 39, amino acid residues wherein one or more or all of A17, V21, N27, K29, P30, R35 (appearing for example in SEQ ID NO: 104), are respectively modified into 17S, 21L, 27S, 29R, 30A, 35V.

In a further particular embodiment, the FR1 and/or FR2 and/or FR3 domains are humanized.

In a further particular embodiment, the VHH molecules may further comprise one or several tags, suitable for e.g., purification, coupling, detection, etc.

Within the context of this invention, the term “tag” includes any peptide sequence which is attached to a polypeptide VHH molecule of the invention to facilitate easy detection or purification of expressed proteins or to identify their binding to RAGE or for site-directed enzymatic chemical/enzymatic conjugation purpose. The tag may be an affinity tag, an epitope tag, a site-specific conjugation tag or a fluorescent tag.

Examples of such tags include a Q-tag which is a tag comprising a glutamine residue inserted in a tag sequence, which is specifically recognized by the TGase, preferably comprising or consisting of sequence LQR, a myc tag (EQKLISEEDL, SEQ ID NO: 38), a poly-His tag (comprising from 2 to 8 histidine residues, preferably 6-8 His residues, e.g., His6 (SEQ ID NO: 37)), a poly-Arg tag (comprising from 2 to 8 arginine residues), a poly-Lys tag (comprising from 2 to 8 lysine residues), an HA tag, a FLAG tag or a GFP tag, a CBP tag, a Strep II tag, a sortase-tag, a SNAP-tag or a combination thereof.

Typically, the one or several tags are located at the C-terminal end of the VHH.

In a further particular embodiment, the VHH molecules may further comprise one or several linkers.

Within the context of this invention, the term “linker” or “spacer” includes one or more amino acid residues, typically from 1 to 10 amino acid residues, used for linking between the VHH molecule of the invention and a tag, or between various tags as described herein, provided that the linker does not specifically bind to the target protein which is RAGE. The linker may be any amino acid residue for example, glycine (Gly), alanine (Ala), serine (Ser), cysteine (Cys), leucine (Leu), asparagine (Asn), lysine (Lys), etc., or a combination thereof. Such a peptide linker is different from conjugation linkers which may be introduced between VHH and the compound of interest, such as bi- or multifunctional agents containing alkyl, aryl or peptide groups by esters, aldehydes or alkyl or aryl acids, anhydride, sulfhydryl or carboxyl groups, groups derived from cyanogen bromide or chloride, carbonyldiimidazole, succinimide esters or sulfonic halides.

As a further illustration, the VHH of the invention may comprise a Gly linker, preferably located at the C-terminal end of the VHH. The Gly linker may comprise a Gly repeat of e.g., 2-7 Gly residues, such as 3, 4 (SEQ ID NO: 39), 5 (SEQ ID NO: 40) or 6 (SEQ ID NO: 41). Specific examples of Gly linkers include Gly3, Gly4 (SEQ ID NO: 39), Gly5 (SEQ ID NO: 40) or SerGlySerGly5 (SEQ ID NO: 41).

In a particular embodiment, VHH of the invention may comprise a Gly linker and a Q-tag, preferably located C-terminally. More specific examples of such VHH comprise the following structure: VHH-Gly linker-Q-tag, wherein the Gly linker comprises, or consists of, 2-6 Gly residues, and the Q-tag preferably contains, or consists of, LQR.

As an illustration, the VHH may comprise, at the C-terminal end, the following additional sequence AAAEQKLISEEDLNGAAHHHHHHGS (SEQ ID NO: 36), wherein simple underline is a myc tag and double underline is a His6 tag (the remaining residues being linkers such as an Ala linker AAA or resulting from cloning).

As another illustration, the VHH may comprise, at the C-terminal end, the following additional sequence GGGGSCHHHHHH (SEQ ID NO: 74), wherein simple underline is a spacer, bold C is a free cysteine available for site-directed chemical conjugation and double underline is a His tag (the remaining residues being linkers or resulting from cloning).

As already taught herein above, specific examples of such tagged RAGE-binding VHH molecules of the invention are molecules comprising or consisting of an amino acid sequence selected from any one of SEQ ID NOs: 31 (VHH 1), 32 (VHH 2), 33 (VHH 3), 34 (VHH 4) and 35 (VHH 5), wherein x is 1.

As another illustration, the VHH of the invention may comprise a Q-tag preferably comprising or consisting of sequence LQR, preferably located at the C-terminal end of the VHH.

As a further illustration, the VHH may comprise, at the C-terminal (“C-ter”) end, the following additional sequence GGGLQR (SEQ ID NO: 42) wherein underlined is a Q-tag and bold is a Gly linker. Other examples are GGGGLQR (SEQ ID NO: 43), GGGGGLQR (SEQ ID NO: 44), GGGGGGLQR (SEQ ID NO: 45) and GGGGGGGLQR (SEQ ID NO: 46).

In a preferred aspect of the invention, the VHH comprises the additional sequence of SEQ ID NO: 42.

In a further particular embodiment, the VHH of the invention may comprise an Ala linker, a His tag, a Gly linker and a Q-tag. Preferably, the linkers and tags are located C-terminally of the VHH. In other embodiments, the Q-tag at least may be located N-terminal (“N-ter”) of the VHH. More specific examples of such VHH comprise the following structure: VHH-Ala linker-His tag-Gly linker-Q-tag, wherein the Ala linker comprises preferably 3 residues, the His tag comprises 2-7 His residues, preferably 6 His residues, the Gly linker comprises 2-6 Gly residues, preferably 3 residues, and the Q-tag preferably contains or consists of LQR.

As another illustration, the VHH may comprise, at the C-ter end, the following additional sequence AAAHHHHHHGGGLQR (SEQ ID NO: 47) wherein underlined is a Q-tag, bold are an Ala and a Gly linker, double underlined is a His tag. Other examples are AAAHHHHHHGGGGLQR (SEQ ID NO: 48), AAAHHHHHHGGGGGLQR (SEQ ID NO: 49), AAAHHHHHHGGGGGGLQR (SEQ ID NO: 50) and AAAHHHHHHGGGGGGGLQR (SEQ ID NO: 51).

In a preferred aspect of the invention, the VHH comprises the additional sequence of SEQ ID NO: 47 (AAAHHHHHHGGGLQR).

Further specific examples of RAGE-binding VHH molecules of the invention are VHH molecules which competitively inhibit binding of a VHH as defined above to a human and a non-human RAGE. The term “competitively inhibits” indicates that the VHH can reduce or inhibit or displace the binding of a said reference VHH to RAGE, in vitro or in vivo. Competition assays can be performed using standard techniques such as, for instance, competitive ELISA or other binding assays. Typically, a competitive binding assay involves a recombinant lung cell or membrane preparation expressing RAGE, optionally bound to a solid substrate, an unlabeled test VHH (or a phage expressing the same) and a labeled reference VHH (or a phage expressing the same). Competitive inhibition is measured by determining the amount of labeled VHH bound in the presence of the test VHH. Usually the test VHH is present in excess, such as about 5 to 500 times the amount of reference VHH. Typically, for ELISA, the test VHH is in 100-fold excess. When a test VHH present in excess inhibits or displaces at least 70% of the binding of the reference VHH to RAGE, it is considered as competitively inhibiting said reference VHH. Preferred competing VHH bind epitopes that share common amino acid residues.

As shown in the experimental section, VHH molecules are able to bind RAGE in vitro and in vivo. They show adequate affinity, with an apparent Kd comprised between 0.1 nM and 10 μM, particularly between 1 nM and 1 μM. Furthermore, preferred molecules (such as those including VHH 1, VHH 2 and VHH 5) bind both human and murine RAGE. Moreover, binding of said VHH of the invention to a human RAGE receptor does not compete with binding of the endogenous RAGE (natural) ligand(s), and thus does not affect regular functions of said ligand. Conjugates produced with such VHH molecules have further been shown to bind RAGE in vitro and to accumulate in the lung and/or in lung cells. Such VHH thus represent potent agents for targeting and drug delivery to the lung.

The VHH of the invention can be synthesized by any technique known to those skilled in the art (chemical, biological or genetic synthesis, etc.). They can be preserved as-is, or be formulated in the presence of a substance of interest or any acceptable excipient.

For chemical syntheses, commercial apparatuses that can incorporate natural as well as non-natural amino acids, such as D enantiomers and residues with side chains with hydrophobicities and steric obstructions different from those of their natural homologues (so-called exotic, i.e., non-coded, amino acids), or a VHH sequence containing one or more peptidomimetic bonds that can include notably intercalation of a methylene (—CH₂—) or phosphate (—PO₂—) group, a secondary amine (—NH—) or an oxygen (—O—) or an N-alkylpeptide, are used.

During synthesis, it is possible to introduce various chemical modifications, such as for example, putting in the N-term or C-term position or on a side chain a lipid (or phospholipid) derivative or a constituent of a liposome or a nanoparticle, in order to be able to incorporate the VHH of the invention within a lipid membrane such as that of a liposome composed of one or more lipid layers or bilayers, or of a nanoparticle. Liposome and nanoparticle are examples of a “vehicle” which may be conjugated with one or more VHH molecules of the invention.

The VHH of the invention can also be obtained from a nucleic acid sequence coding for the same, as described further below in SEQ ID NOs: 21 to 30 (cf. Table 3).

### Conjugates

A further object of the invention relates to conjugates (also interchangeably called herein “chimeric agents”) comprising one or more VHH molecules as defined above, conjugated to at least one additional compound, in particular to at least one additional molecule, agent or compound of interest, for example to a scaffold of interest.

This additional compound may be a distinct VHH or a molecule which is not a VHH.

The at least one additional molecule, agent or compound of interest may be any molecule, agent or compound such as a stabilizing group (also interchangeably called herein “a half-life extending moiety” or “a scaffold”), a therapeutic (i.e., active) compound, medicament or drug, a diagnostic agent, an imaging compound, a tracer, etc., or a vehicle comprising such a therapeutic, diagnostic or imaging compound.

In a particular aspect, the chimeric agent/conjugate may comprise both kind of additional compounds, i.e. i) a stabilizing group, a half-life extending moiety or scaffold and ii) a therapeutic, diagnostic or imaging compound, or a vehicle comprising the same.

The stabilizing group or a half-life extending moiety increases the plasma half-life of the VHH or conjugate.

The therapeutic compound is for example selected from a peptide, a polypeptide, a protein, an antibody, a nucleic acid and any fragment thereof.

Examples of conjugated molecules, agents or compounds of interest include, without limitation, any chemical entity such as a small chemical molecule (for example a chelating agent, an antibiotic, antiviral, immunomodulator, antineoplastic, anti-inflammatory, or adjuvant, etc.); a peptide, polypeptide or protein (for example an enzyme, hormone, cytokine, apolipoprotein, growth factor, antigen, antibody or part of an antibody, adjuvant, etc.); a nucleic acid (for example a RNA or a DNA, of human, viral, animal, eukaryotic, prokaryotic, plant or synthetic origin, etc., including e.g., coding genes, inhibitory nucleic acids such as ribozymes, antisense oligonucleotides (ASOs), interfering nucleic acids (siRNAs), small activating RNAs (saRNAs), mRNAs, full genomes or portions thereof, plasmids, etc.); a lipid (nano) particle, a Cell-Derived Vesicle (CDV) such as an exosome, a virus, a marker, or a tracer, for instance. Generally, the “molecule, agent or compound of interest” can be any drug (active) ingredient, whether a chemical, biochemical, natural or synthetic compound. Generally, the expression “small chemical molecule, agent or compound” designates a molecule of pharmaceutical interest with a maximum molecular weight of 1000 Daltons, typically between 300 Daltons and 700 Daltons.

The vehicle may be selected for example from a virus, a virus-like particle (VLP), a Cell-Derived Vesicle (CDV), an exosome, a lipid vehicle and a polymer vehicle, and is preferably a lipid nanoparticle (LNP), a micelle or a liposome.

The conjugated compound is typically a medicament (such as a small drug, nucleic acid or polypeptide, e.g., an antibody or fragment thereof) or an imaging agent suitable for treating or detecting a lung disease such as, for example an infectious, immunological or cancerous lung pathology.

The chimeric agent may also contain, in addition to or instead of said compound of interest, a stabilizing group to increase the plasma half-life of the VHH or conjugate. Particular chimeric agents of the invention thus comprise i) at least one VHH, for example several VHH molecules, ii) a stabilizing group, iii) a compound of interest, typically a therapeutic, diagnostic or imaging compound, and optionally iv) a vehicle, in any order.

In a particular aspect herein described, the compound of interest is at the same time a group allowing the stabilization of the VHH molecule(s) of the invention.

The stabilizing group may be any group known to have substantial plasma half-life (e.g. at least 1 hour, at least 1 day or at least 1 week) and essentially no adverse biological activity. Examples of such stabilizing group include, for instance, an antibody or a fragment thereof such as a Fc fragment of an immunoglobulin, a VHH molecule or variants thereof, large human serum proteins such as albumin, in particular human serum albumin (HSA), or serum albumin binding molecules or IgGs or PEGs molecules.

In a particular embodiment, the stabilizing group is a Fc fragment. More preferably, the stabilizing group is an Fc fragment of an IgG1 such as a Fc fragment of a human IgG1.

In another particular embodiment, the conjugate according to the invention comprises a stabilizing group which is an albumin-binding moiety that preferably binds to the albumin with an affinity from about 1 nM to about 10 μM, thus improving the pharmacokinetic profile of the compound of interest by a progressive release of the conjugate from the albumin. Such albumin-binding moieties include e.g., a fragment of Evans blue (EB) dye, fatty acids and derivatives thereof such as the C16 group and the 4-(p-iodophenyl) butytryl (PIB) group, 89D03 peptide, and ABD035 protein (a 46-residues three-helix bundle albumin binding domain).

The VHH may be conjugated in N-ter or C-ter of the stabilizing group, or both. When the stabilizing group is a Fc fragment, conjugation is typically by genetic fusion. The resulting protein may remain as a monomeric agent, or multimerize, depending on the nature of the stabilizing group. In the case of a Fc fragment, the fusion protein Fc-VHH or VHH-Fc usually forms homodimers.

In the conjugate compounds of the invention, coupling can be performed by any acceptable means of bonding taking into account the chemical nature, obstruction and number of conjugated entities. Coupling can thus be carried out by one or more covalent, ionic, hydrogen, hydrophobic or Van der Waals bonds, cleavable or non-cleavable in physiological medium or within cells, preferably cleavable in particular when the present invention is used in the context of the delivery of at least one active agent on the lung site. Furthermore, coupling can be made at various reactive groups, and notably at one or more terminal ends and/or at one or more internal or lateral reactive groups. Coupling can also be carried out using genetic engineering.

It is preferable that the interaction is sufficiently strong so that the VHH is not dissociated from the active substance before having reached its site of action (i.e., the lung site). For this reason, the preferred coupling of the invention is covalent coupling, although non-covalent coupling may also be employed. The compound of interest can be coupled with the VHH either at one of the terminal ends (N-term or C-term), or at a side chain of one of the constitutive amino acids of the sequence (Majumdar S. and Siahaan T J., “Peptide-mediated targeted drug delivery”. Med Res Rev., 2012 May; 32(3):637-58). The compound of interest can be coupled directly to a VHH, or indirectly by means of a conjugation linker or spacer. Means of covalent chemical coupling, calling upon a spacer or not, include for instance those selected from bi- or multifunctional agents containing alkyl, aryl, thiols or peptide groups by esters, aldehydes or alkyl or aryl acids, anhydride, sulfhydryl or carboxyl groups, groups derived from cyanogen bromide or chloride, carbonyldiimidazole, succinimide esters or sulfonic halides.

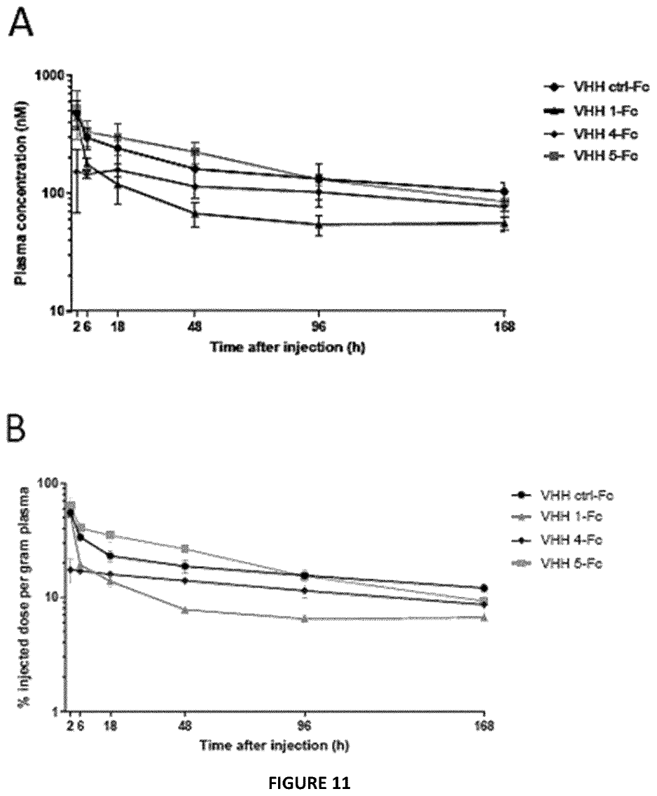

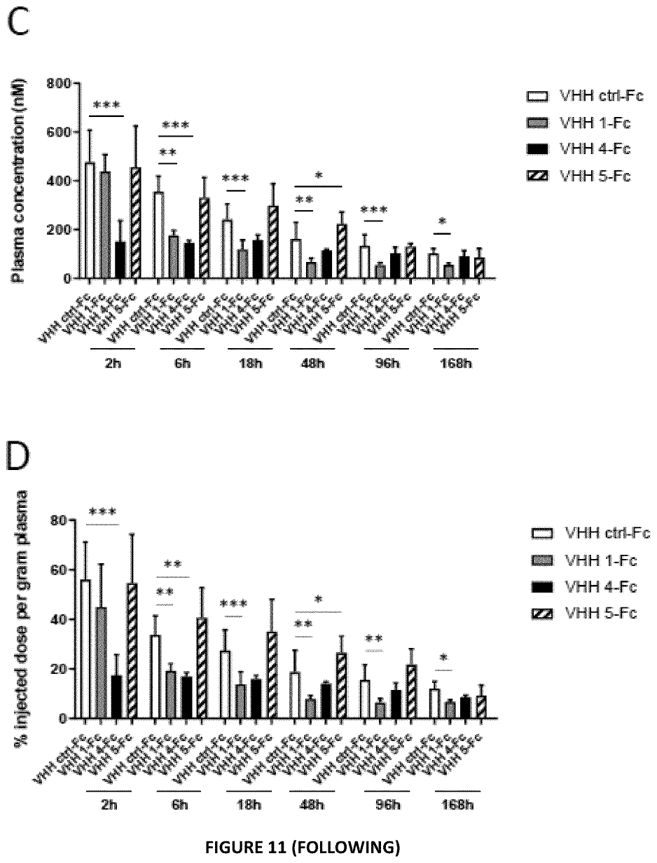

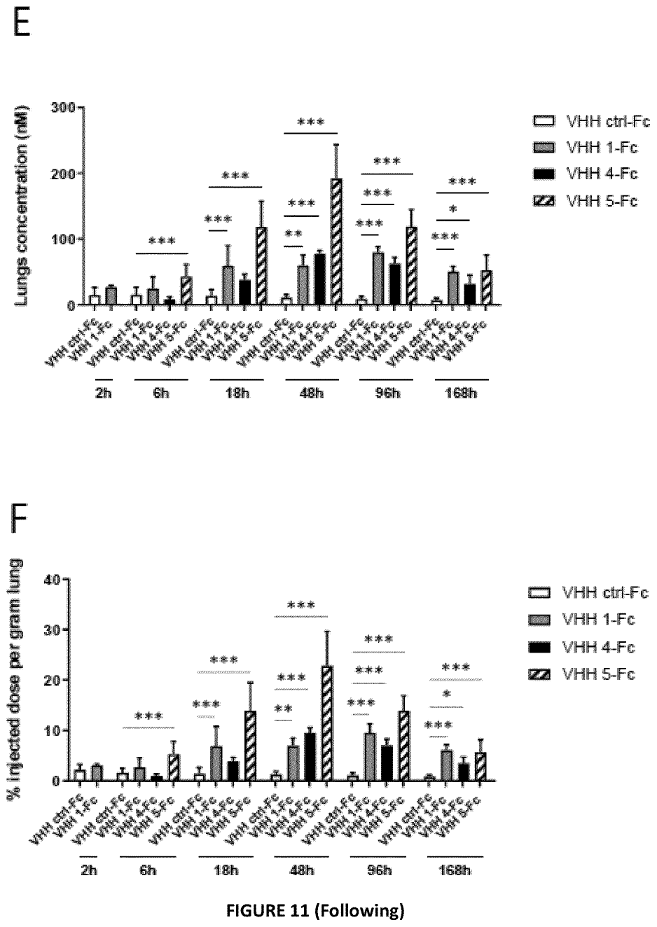

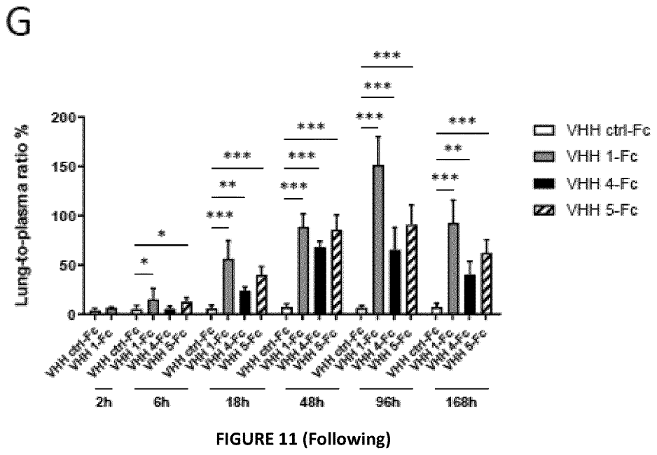

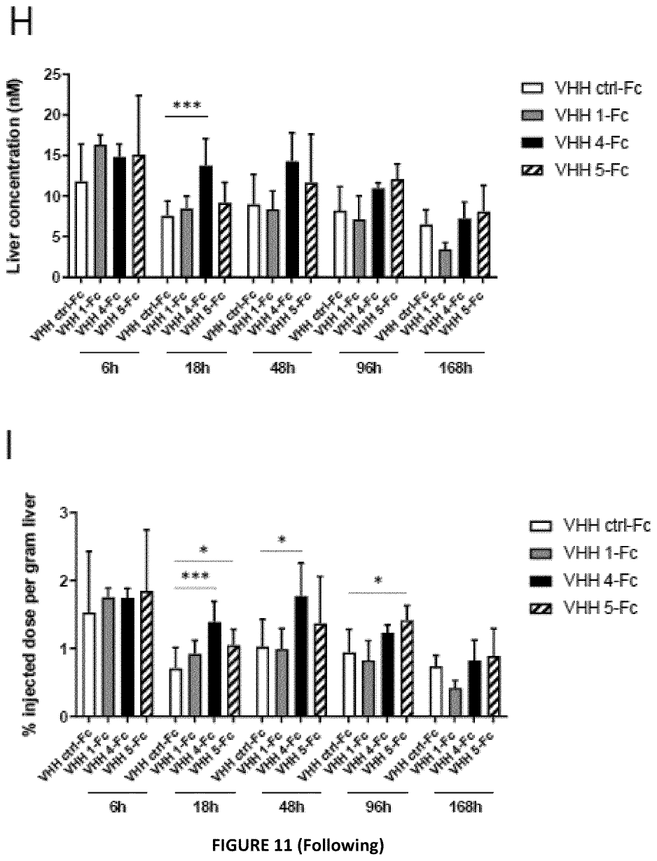

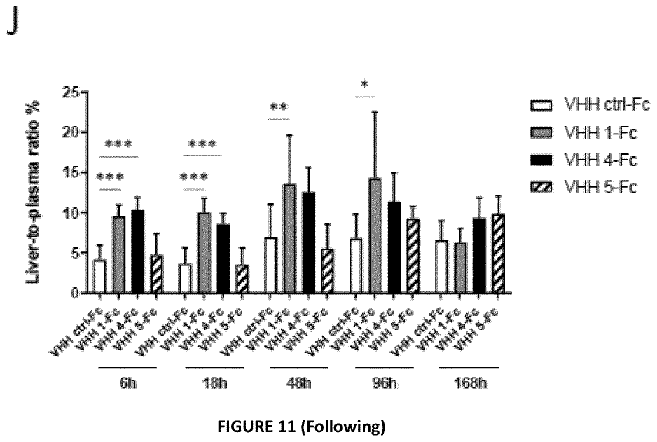

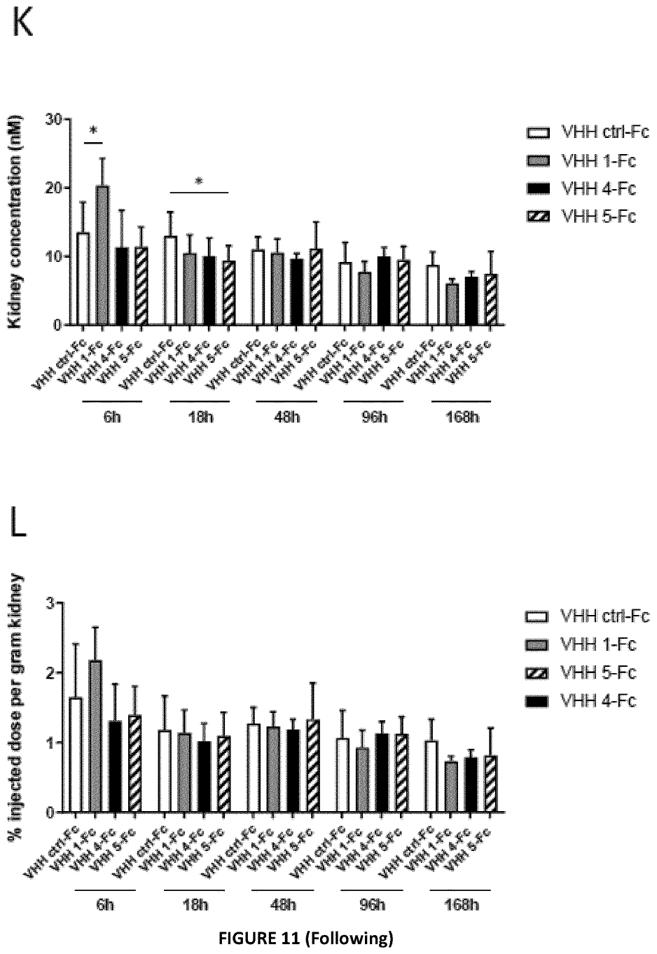

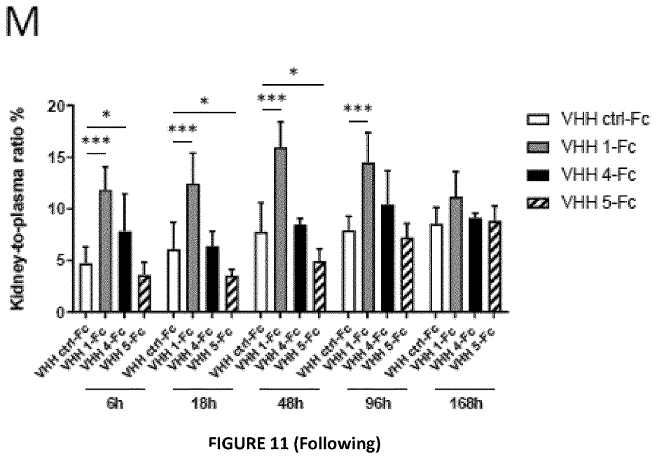

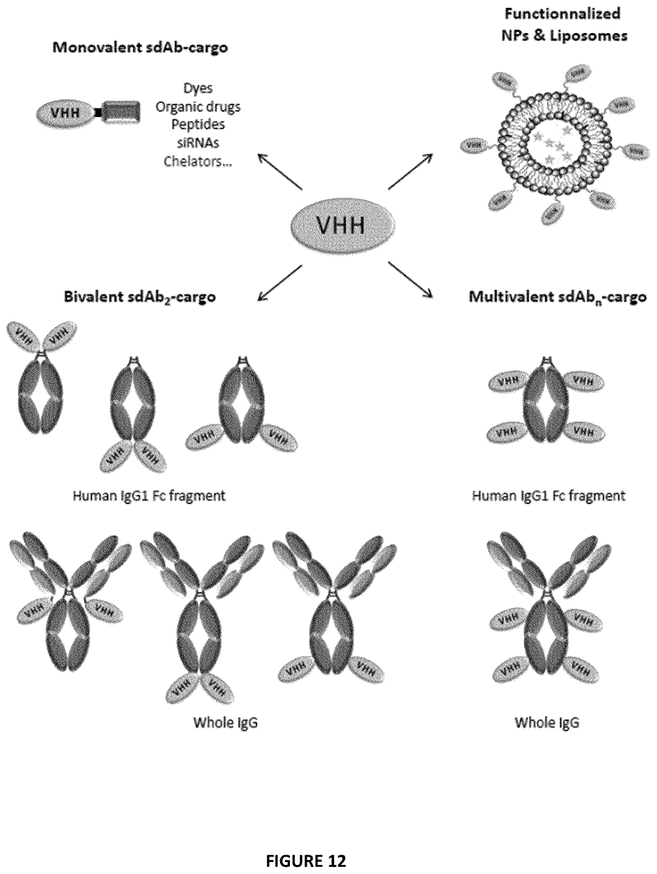

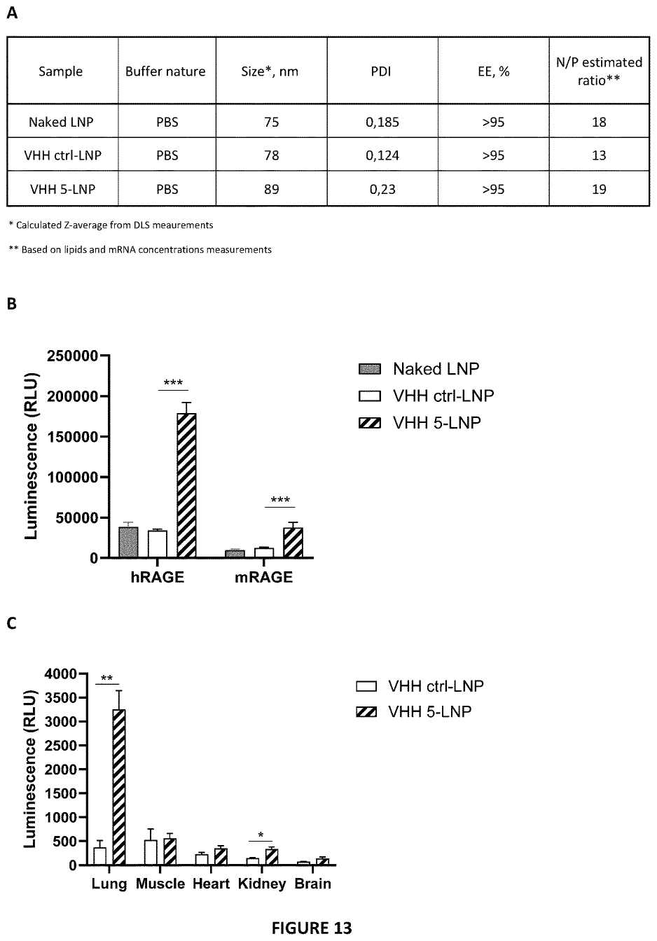

Illustrative strategies for conjugating a VHH of the invention to a molecule or scaffold are disclosed in FIG. 11.

In a particular embodiment, coupling (or conjugation) is by genetic fusion. Such strategy can be used when the coupled molecule is a peptide or polypeptide. In such a case, a nucleic acid molecule encoding the VHH fused to the molecule is prepared and expressed in any suitable expression system, to produce the conjugate.

In another particular embodiment, coupling (or conjugation) is performed using the thiol/maleimide chemistry technology. For this reaction to occur, the VHHs produced with the additional peptidic tag GGGGSCHHHHHH (SEQ ID NO: 74) fused to their C-terminus are typically used.

Since VHHs only contain cysteins engaged in a disulfide bridge, the additional cystein introduced in the tag is the only one to be chemically reactive towards maleimides. It allows the specific conjugation of the VHH with maleimide-derivatized molecules of interest. The reaction proceeds in two steps. First the VHH-GGGGSCHHHHHH is reduced using a mild reducing agent such as 2-MEA (2-mercaptoethanol), TCEP (tris(2-carboxyethyl)phosphine) or DTT (dl-1,4-dithiothreitol). During the production process of the VHH-GGGGSCHHHHHH, the additional cystein present in the tag may react with a cystein borne by another VHH-GGGGSCHHHHHH leading to a mix of free VHH-GGGGSCHHHHHH and dimers of VHH-GGGGSCHHHHHH. In a second step, the VHH-GGGGSCHHHHHH is allowed to react at pH in the range 6.5-7.5 with maleimide-functionalized molecules of interest to form VHH-molecules conjugates linked in a covalent and stable manner.

In another particular embodiment, coupling (or conjugation) is by enzymatic reaction. In particular, site-specific conjugation onto the VHH can be performed using the transglutaminase enzyme (TGase). TGase catalyzes the formation of a stable isopeptidic bond between (i) the side chain of a glutamine residue inserted in a tag sequence specifically recognized by the TGase (namely a Q-tag) and (ii) an amino-functionalized donor substrate. In this regard, the inventors have developed a particular tag sequence (named “Q-tag”) which is recognized by TGase and may be used to couple VHH of the invention to any molecule of interest, particularly chemical drugs or agents. For this purpose, VHHs are prepared by genetic fusion to add in tandem (typically to their C-terminus) the following tags: first an optional trialanine linker, then an optional His-tag, then an optional small triglycine linker, and finally a Q-tag. The triglycine linker allows to space out the Q-tag to allow a better accessibility of the TGase to the glutamine while the His tag aims at facilitating the purification of the VHH and its further functionalized versions.

The general conjugation strategy that was developed is a convergent synthesis that is based on a process comprising:
1) introduction onto the glutamine of the Q-tag of the VHH a reactive moiety for further conjugation to a molecule of interest. In this objective, a heterobifunctional conjugation linker having two different reactive ends is allowed to be processed by the TGase: one suitable primary amine-group toward the TGase and one orthogonal reactive moiety. Representative examples of such orthogonal and reactive groups include azides, constraints alkynes such as DBCO (dibenzocyclooctyne) or BCN (bicyclo[6.1.0]nonyne), tetrazines, TCO (trans-cyclooctene), free or protected thiols, etc.
2) introduction onto the molecule of interest of a reactive moiety complementary to the one incorporated onto the VHH Q-tag. Representative examples of such orthogonal and reactive groups include azides, constrained alkynes such as DBCO or BCN, tetrazines, TCO, free or protected thiols, etc.
3) conjugation of both the functionalized VHH and molecule owing to their complementary reactive groups.

Herein described is also a method for coupling two molecules using a Q-tag as defined above through TGase coupling reaction. A further object of the invention is a VHH of the invention comprising a Q-tag. A further object of the invention is a VHH molecule of the invention comprising a linker, such as a Gly linker, and a Q-tag.

Preferred VHH of the invention have the following structure:

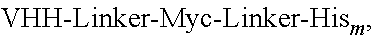

wherein:
VHH is any VHH molecule;
Linker is any molecular linker such as an Ala or Gly linker (preferably the two linkers are different); and
m is an integer from 0 to 8, preferably m is 6 or 8.

In a particular embodiment, the invention relates to a conjugate comprising a VHH covalently linked to a chemical entity. Preferred variants of such conjugates contain one (1) VHH and one (1) chemical entity.

In another particular embodiment, the invention relates to a conjugate comprising a VHH covalently linked to a nucleic acid. The nucleic acid may be an antisense oligonucleotide (“ASO”), a ribozyme, an aptamer, a mRNA, a siRNA, etc. Preferred variants of such conjugates contain one VHH and one nucleic acid molecule.

In another particular embodiment, the invention relates to a conjugate comprising a VHH covalently linked to a peptide. The peptide may be an active molecule, a bait, a tag, a ligand, etc. Preferred variants of such conjugates contain one VHH and one peptide.

In another embodiment, the invention relates to a conjugate comprising a VHH covalently linked to a dye.

In another embodiment, the invention relates to a conjugate comprising a VHH covalently linked to a nanoparticle and/or liposome, for example a lipidic particle or nanoparticle (“LNP”). The nanoparticle and/or liposome may be loaded or functionalized with active agents. Preferred variants of such conjugates contain several VHH molecules coupled to each nanoparticle or liposome.

In a further embodiment, the conjugate comprises an antibody or a fragment thereof to which one or several VHH molecules are coupled.

Typically, a VHH molecule is coupled to a C- or N-terminal end of a heavy or light chain, or both, or to the C- or N-terminal end of an Fc fragment. In a preferred aspect, the VHH molecule is coupled to the N-terminal end of a heavy chain. In an even more preferred aspect, the VHH molecule is coupled to the C-terminal end of a heavy chain.

In another particular aspect, the conjugate comprises, or consists of, a single VHH molecule coupled to an antibody fragment which may be a heavy chain or a light chain. In this aspect, the VHH molecule is indifferently coupled to the C-terminal end or to the N-terminal end of the chain. Preferably, it is coupled to the C-terminal end.

Inventors also herein describe a method for preparing a conjugate compound such as defined above, characterized in that it comprises a step of coupling a VHH and a molecule or scaffold, preferably by a chemical, biochemical or enzymatic pathway, or by genetic engineering.

In a chimeric agent of the invention, when several VHHs are present, they may bind to similar or different binding domains.

### Nucleic Acids, Vectors and Host Cells

A further aspect of the invention relates to a nucleic acid encoding a VHH as defined above, or a conjugate thereof (when the conjugated moiety is an amino acid sequence). The nucleic acid may be single- or double-stranded. The nucleic acid can be a DNA (for example a cDNA or a gDNA), a RNA (for example a mRNA or a gRNA), or a mixture thereof. It can be in single stranded form or in duplex form, or it can be a mixture of the two. It can comprise modified nucleotides, comprising for example a modified bond, a modified purine or pyrimidine base, or a modified sugar. It can be prepared by any method known to one of ordinary skill in the art, including chemical synthesis, recombination and/or mutagenesis. The nucleic acid according to the invention may be deduced from the amino acid sequence of the VHH molecules according to the invention and codon usage may be adapted according to the host cell in which the nucleic acid shall be transcribed. These steps may be carried out according to methods well known to one of ordinary skill in the art and some of which are described in the reference manual Sambrook et al. (Sambrook J, Russell D (2001) Molecular cloning: a laboratory manual, Third Edition Cold Spring Harbor).

Specific examples of such nucleic acid sequences include the sequences comprising any one of SEQ ID NOs: 21, 23, 25, 27 or 29, and the complementary sequence thereto, as well as fragments thereof devoid of or including the optional tag-coding portion appearing in SEQ ID Nos: 22, 24, 26, 28 or 30. The domains encoding CDR1 (SEQ ID NO: 52-56), CDR2 (SEQ ID NO: 57-61) and CDR3 (SEQ ID NO: 62-66) are underlined. The tag-coding portion appears in bold in Table 3 (SEQ ID NO: 67: GCGGCCGCAGAACAAAAACTCATCTCAGAAGAGGATCTGAATGGGGCCG CACATCACCACCATCACCATGGGAGCTAG).

The invention also relates to a vector containing such a nucleic acid, optionally under control of regulatory sequences (e.g., promoter, terminator, etc.). The vector may be a plasmid, virus, cosmid, phagemid, artificial chromosome, etc. In particular, the vector may comprise a nucleic acid of the invention operably linked to a regulatory region, i.e. a region comprising one or more control sequences. Optionally, the vector may comprise several nucleic acids of the invention operably linked to several regulatory regions.

The term “control sequences” means nucleic acid sequences necessary for expression of a coding region. Control sequences may be endogenous or heterologous. Well-known control sequences and currently used by the person skilled in the art will be preferred. Such control sequences include, but are not limited to, promoter, signal-peptide sequence and transcription terminator.

The term “operably linked” means a configuration in which a control sequence is placed at an appropriate position relative to a coding sequence, in such a way that the control sequence directs expression of the coding region.

The present invention further relates to the use of a nucleic acid or vector according to the invention to transform, transfect or transduce a host cell or to produce a composition, including a pharmaceutical composition, to transform, transfect or transduce a host cell.

The present invention also provides a host cell comprising one or several nucleic acids of the invention and/or one or several vectors of the invention.

The term “host cell” also encompasses any progeny of a parent host cell that is not identical to the parent host cell due to mutations that occur during replication. Suitable host cells may be prokaryotic (e.g., a bacterium) or eukaryotic (e.g., yeast, plant, insect or mammalian cell). Specific illustrative examples of such cells include E. coli strains, CHO cells, Saccharomyces strains, plant cells, sf9 insect cells etc.

### Uses

VHH molecules of the invention can bind to RAGE and thus target/deliver molecules to RAGE-expressing lung cells.

Within the context of this invention, binding is preferably specific, so that binding to RAGE occurs with higher affinity than binding to any other antigen in the same species. Preferred VHH molecules of the invention bind human RAGE and murine RAGE.

The invention thus relates to methods of targeting/delivering a compound to/through a RAGE-expressing lung cell, comprising coupling said compound to at least one VHH of the invention.

The invention further relates to the use of a VHH such as defined above, as a vector for the transport of a compound to/through a RAGE-expressing lung cell.

The invention also relates to the use of a VHH such as defined above for preparing a drug (/medicament) capable of addressing the lung site.

The invention also relates to a method for enabling or improving the addressing of a compound of interest to the lung site, comprising the coupling of the compound to a VHH molecule of the invention.

As explained herein above, the VHH of the invention may be used to transport or deliver any compound, such as for example chelating agents, small drugs, amino acids, peptides, polypeptides, proteins, lipids, nucleic acids, viruses, liposomes, exosomes etc. to the lung.

A vehicle may be used to transport or deliver the conjugate (including the VHH) such as for example a virus, a virus-like particle (VLP), a Cell-Derived Vesicle (CDV), an exosome, a lipid vehicle or a polymer vehicle, and is preferably a lipid nanoparticle (LNP), a micelle or a liposome.

The invention also relates to a pharmaceutical composition, in particular a diagnostic or therapeutic composition, characterized in that it comprises at least one VHH or chimeric (/conjugate) compound, associated to, or present in, a vehicle or not, for example, in the context of a therapeutic composition, a VHH-drug conjugate, such as defined above and one or more pharmaceutically acceptable supports, carriers or excipients. The invention also in particular relates to a diagnostic composition characterized in that it comprises a VHH or chimeric (/conjugate) compound, associated to, or present in, a vehicle or not, for example a VHH-diagnostic or medical imaging agent conjugate compound, such as defined above.

The conjugate can be used in the form of any pharmaceutically acceptable salt. The expression “pharmaceutically acceptable salts” refers to, for example and in a non-restrictive way, pharmaceutically acceptable base or acid addition salts, hydrates, esters, solvates, precursors, metabolites or stereoisomers, said vectors or conjugates loaded with at least one substance of interest.

The expression “pharmaceutically acceptable salts” refers to nontoxic salts, which can be generally prepared by reacting a free base with a suitable organic or inorganic acid. These salts preserve the biological effectiveness and the properties of free bases. Representative examples of such salts include water-soluble and water-insoluble salts such as acetates, N-methylglucamine ammonium, amsonates (4,4-diaminostilbene-2,2′-disulphonates), benzenesulphonates, benzonates, bicarbonates, bisulphates, bitartrates, borates, hydrobromides, bromides, buryrates, camsylates, carbonates, hydrochlorates, chlorides, citrates, clavulanates, dichlorhydrates, diphosphates, edetates, calcium edetates, edisylates, estolates, esylates, fumarates, gluceptates, gluconates, glutamates, glycolylarsanylates, hexafluorophosphates, hexylresorcinates, hydrabamines, hydroxynaphthoates, iodides, isothionates, lactates, lactobionates, laurates, malates, maleates, mandelates, mesylates, methylbromides, methylnitrates, methylsulphates, mucates, napsylates, nitrates, 3-hydroxy-2-naphthoates, oleates, oxalates, palmitates, pamoates (1,1-methylene-bis-2-hydroxy-3-naphtoates, or emboates), pantothenates, phosphates, picrates, polygalacturonates, propionates, p-toluenesulphonates, salicylates, stearates, subacetates, succinates, sulphates, sulphosalicylates, suramates, tannates, tartrates, teoclates, tosylates, triethiodides, trifluoroacetates and valerianates.

The compositions of the invention advantageously comprise a pharmaceutically acceptable support, carrier or excipient. The pharmaceutically acceptable support, carrier or excipient can be selected from the carriers classically used according to each mode of administration. According to the mode of administration envisaged, the compounds can be in solid, semi-solid or liquid form. For solid compositions such as tablets, pills, powders, or granules that are free or are included in gelatin capsules, the active substance can be combined with: a) diluents, for example lactose, dextrose, sucrose, mannitol, sorbitol, cellulose and/or glycine; b) lubricants, for example silica, talc, stearic acid, its magnesium or calcium salt and/or polyethylene glycol; c) binders, for example magnesium and aluminum silicate, starch paste, gelatin, tragacanth, methylcellulose, sodium carboxymethyl cellulose and/or polyvinylpyrrolidone; d) disintegrants, for example starch, agar, alginic acid or its sodium salt, or effervescent mixtures; and/or d) absorbents, dyes, flavoring agents and sweeteners. The excipients can be, for example, mannitol, lactose, starch, magnesium stearate, sodium saccharin, talc, cellulose, glucose, sucrose, magnesium carbonate and analogues of pharmaceutical quality. For semi-solid compositions such as suppositories, the excipient can, for example, be an emulsion or oily suspension, or polyalkylene glycol-based, such as polypropylene glycol. Liquid compositions, in particular injectables or those included in a soft capsule, can be prepared, for example, by dissolution, dispersion, etc., of the active substance in a pharmaceutically pure solvent such as, for example, water, physiological saline solution, aqueous dextrose, glycerol, ethanol, oil and analogues thereof.

The compositions or conjugates of the invention can be administered by any suitable route and, in a non-restrictive way, by parenteral route, such as, for example, in the form of preparations that can be injected by subcutaneous, intravenous or intramuscular route; by oral route (or per os), such as, for example, in the form of coated or uncoated tablets, gelatin capsules, powders, pellets, suspensions or oral solutions (one such form for oral administration can be either with immediate release or with extended or delayed release); by rectal route such as, for example, in the form of suppositories; by topical route, in particular by transdermal route, such as, for example, in the form of patches, pomades or gels; by intranasal route such as, for example, in aerosol and spray form; by perlingual route; or by intraocular route.

Preferably, the VHH or conjugate of the invention, or composition comprising such a VHH or conjugate, is administered by intravenous or subcutaneous route.

The pharmaceutical compositions of the invention typically comprise an effective dose of a VHH or conjugate of the invention. A “therapeutically effective dose” as described herein refers to the dose that gives a therapeutic effect for a given condition and administration schedule. It is typically the average dose of an active substance to administer to appreciably improve some of the symptoms associated with a disease or a pathological state. For example, in treating a cancer of the lung, a pathology, a lesion or a disorder affecting the lungs, the dose of an active substance that decreases, prevents, delays, eliminates or stops one of the causes or symptoms of the disease or disorder would be therapeutically effective. A “therapeutically effective dose” of an active substance does not necessarily cure a lung disease or disorder but will provide a treatment for this disease or disorder so that its appearance is delayed, impeded or prevented, or its symptoms are attenuated, or its term is modified or, for example, is less severe, or the recovery of the patient is accelerated.

The “therapeutically effective dose” of a VHH or conjugate of the invention is for example of about 1 mg to about 100 mg per kilo of body weight of the subject who will be administered with said VHH or conjugate.

It is understood that the “therapeutically effective dose” for a person in particular will depend on various factors, including the activity/effectiveness of the active substance, its time of administration, its route of administration, its toxicity, its rate of elimination and its metabolism, drug combinations/interactions and the severity of the disease (or disorder) treated on a preventive or curative basis, as well as the age, weight, overall health, sex and/or diet of the patient.

In the context of imaging or diagnostic, the exposure levels are proportional to the activity of the selected radioactive tracer at the time of injection and depend on the length of stay in the body (until its physical or biological removal). They obviously depend on the selected tracer. Activity at the time of injection is evaluated in millions of Becquerels (MBq or mega-Becquerels). However, many practitioners still rely on traditional millicuries, unit still widely used (1 mCi equals 37 MBq).

The injected activities vary greatly depending on the exam, as for example from 1 mCi for kidney scintigraphy with iodine-123 to about 27 mCi (1000 MBq) for cardiac scintigraphy with technetium. The exposure is naturally proportional to the injected activity but also on the irradiating power of the radioactive substance. This irradiating character depends on the diagnostic or therapeutic use, on the nature of radiation, specific activity, how long it stays in the body, and how the radioactive isotope is distributed in the patient body. It varies widely.

Thus, the pharmaceutically effective dose of a VHH or conjugate of the invention for use in imaging or diagnostic is for example of about 1 mCi to about 40 mCi.

Depending on the substance coupled, the conjugates and compositions of the invention can be used for imaging, diagnosing, preventing and/or treating, pathologies or disorders affecting the lungs, such as for example infectious pathologies, inflammation pathologies such as asthma, bronchial asthma and chronic obstructive pulmonary diseases (COPD) also referred to as emphysema, and/or cancers. The VHH of the invention have the capacity to target RAGE-expressing cells, particularly lung cells and/or to cross lung cell membranes. The RAGE is enriched in lungs compared to distinct organs. RAGE is also expressed in lung endothelial cells.

In this respect, the invention relates to the use of a pharmaceutical conjugate or pharmaceutical composition (in particular a therapeutic composition) as described herein above for preventing or treating lung pathologies or disorders such as, in a non-restrictive manner, a lung tumor (the tumor being a benign tumor or a malignant tumor, i.e. a cancerous tumor), in particular a lung metastatic cancer, or a bacterial, viral, parasitic or fungal infectious pathology of the lung, etc., or any other known lung disease.

In the context of the present invention, the lung cancer is also identified as a lung carcinoma or malignant lung tumor. The lung tumor is for example a non-small cell lung cancer or carcinoma (NSCLC) or a small-cell lung cancer or carcinoma (SCLC). lung cancer or carcinoma (NSCLC) or a small-cell lung cancer or carcinoma (SCLC).

In the context of the present invention, the infectious lung disease is a bacterial infection such as pneumonia or tuberculosis or a viral infection such as an infection by SARS-COV2. The infectious lung disease may also be a parasite or fungus infection. In a particular aspect, the infection is a zoonotic disease caused by a virus, bacteria, parasite or fungus.

In the context of the present invention, the infectious pathology is a parasitic infection such as pneumocystis, pulmonary hydatid disease, porocephaliasis, aspergillosis, paragonimiasis, an infection by penicillum marneffeiis, schistosomiasis, ascariasis, hookworm infestations, filarioses, dirofilariasis, tropical pulmonary eosinophilia, toxocariasis, amoebiasis and malignant tertian malaria.

In the context of the present invention, the infectious pathology is for example a fungal infection caused by endemic fungi or by opportunistic fungi, including aspergillus (possibly responsible for invasive aspergillosis), cryptococcus (possibly responsible for cryptococcosis), pneumocystis (possibly responsible for pneumonia).

In the context of the present invention, the genetic and/or rare diseases affecting lung are for example cystic fibrosis, pulmonary hypertension, interstitial lung diseases including beryllium disease and hypersensitivity pneumonitis, rare lung diseases such as lymphangioleiomyomatosis (LAM), Pulmonary Alveolar Proteinosis (PAP) Syndrome, Hermansky-Pudlak Syndrome (HPS) Birt-Hogg-Dubé Syndrome (BHD, Pulmonary Langerhans Cell Histiocytosis (PLCH), Diffuse Idiopathic Pulmonary Neuroendocrine Cell Hyperplasia (DIPNECH), Pulmonary Alveolar Microlithiasis (PAM), Alpha-1 Antitrypsin Deficiency (Alpha-1), or Generalized Lymphatic Anomaly (GLA) (also known as lymphangiomatosis).

The invention also relates to a VHH, conjugate, or pharmaceutical composition (in particular a diagnostic composition) as described herein above for use for imaging and/or diagnosing a lung pathology or disorder such as a lung tumor (the tumor being a benign lung tumor or a malignant lung tumor), in particular a lung metastatic cancer, a bacterial, viral, parasitic or fungal infectious pathology of the lung, or a genetic and/or rare disease of the lung.

The invention in particular relates to a VHH, conjugate, or pharmaceutical composition as described herein above for use for imaging and/or diagnosing (the presence of) a lung tumor or of lung metastatic cancer cells.

The invention also relates to a VHH, conjugate or pharmaceutical composition such as described above for use for imaging, diagnosing, preventing and/or treating a genetic and/or rare disease such as, in non-restrictive manner, cystic fibrosis, pulmonary hypertension, an interstitial lung disease such as beryllium disease or hypersensitivity pneumonitis, a rare disease such as lymphangioleiomyomatosis (LAM), the Pulmonary Alveolar Proteinosis (PAP) Syndrome, the Hermansky-Pudlak Syndrome (HPS), the Birt-Hogg-Dubé Syndrome (BHD), Pulmonary Langerhans Cell Histiocytosis (PLCH), Diffuse Idiopathic Pulmonary Neuroendocrine Cell Hyperplasia (DIPNECH), Pulmonary Alveolar Microlithiasis (PAM), Alpha-1 Antitrypsin Deficiency (Alpha-1), or GLA/lymphangiomatosis.

The invention also relates to a VHH, conjugate or pharmaceutical composition such as defined herein above, wherein the conjugated agent is or comprises a virus or a virus-like particle, such as a recombinant virus. The invention may indeed be used to increase any RAGE enriched lung tissue delivery of recombinant (e.g., replication-defective or attenuated) viruses used in gene therapy, such as adenoviruses, adeno-associated viruses, lentiviruses, retroviruses, etc., or virus-like particles. Coupling to a virus or VLP may be performed e.g., by coupling to the capsid protein of the virus.

The invention also relates to methods for preventing or treating any of the above conditions or diseases by administering to a subject in need thereof a VHH, conjugate or composition of the invention.

The invention also relates to the use of a VHH, conjugate or composition of the invention for the manufacture of a medicament for treating any of the above conditions or diseases.

Other aspects and advantages of the present invention will become apparent upon consideration of the examples below, which are only illustrative in nature and which do not limit the scope of the present application.

### EXAMPLES

### Example I

### Evaluation of RAGE Expression in Various Tissues.

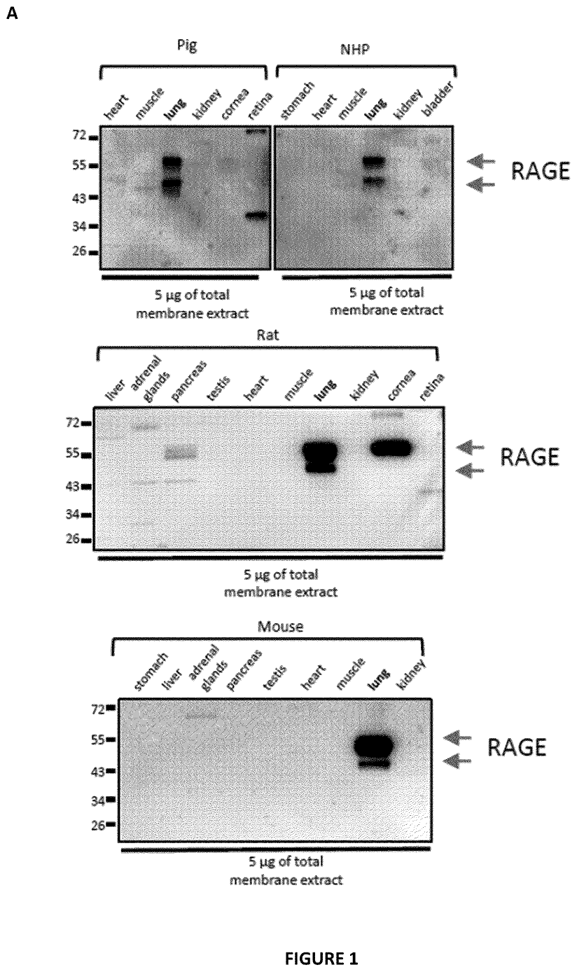

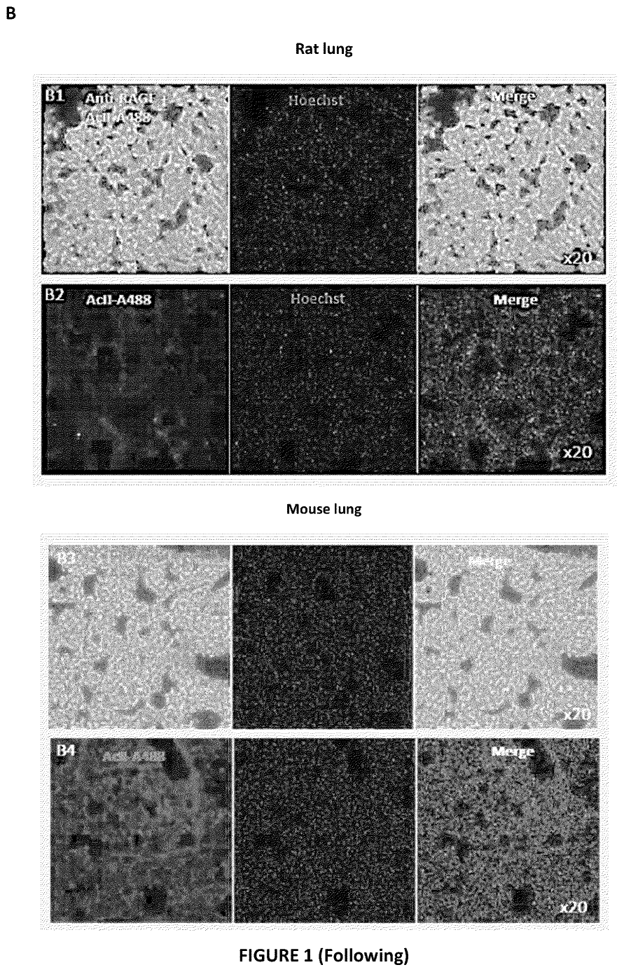

Inventors analyzed by Western Blot the cell membrane expression profile of RAGE in different tissues (FIG. 1-A) of rat, mouse, pig and non-human primate (NHP; rhesus monkey). The ProteoExtract Subcellular Proteome Extraction Kit (Calbiochem, La Jolla, CA, USA) was used to prepare the membrane extracts. Membrane extracts were quantified using the BioRad DC Protein Assay (Bio-Rad, Hercules, CA, USA) following the manufacturer's instructions. Membrane proteins were separated by sodium dodecyl sulfate polyacrylamide gel electrophoresis (SDS-PAGE) on 4-12% polyacrylamide gels and transferred onto nitrocellulose membranes (Thermo Fisher Scientific). Proteins were probed with a primary rabbit polyclonal antibody against RAGE (1/1000, abcam #ab37647) followed by an HRP-conjugated donkey anti-rabbit IgG secondary antibody (Jackson ImmunoResearch, 711-035-152) diluted 1/30,000. Finally, proteins were detected using chemiluminescence (ECL; Cytiva).

As shown in FIG. 1-A, full-length RAGE (~55-kDa) is mainly expressed at a high level in lungs from rat, mouse, pig and non-human primate. RAGE is also expressed in rat cornea and at low or non-detectable levels in the membrane extracts from other tissues: heart, muscle, kidney, retina, bladder, adrenal, pancreas, testis, and stomach. High level expression and tissue distribution of RAGE in lungs of rat and mice was confirmed by immunocytochemistry (FIG. 1-B). For immunohistochemistry, trans-cardiac NaCl 0.9% perfusion was followed by 50 mL of PBS 1×-4% paraformaldehyde (PFA). Mice or rat lungs were then removed and snap frozen on dry ice in mounting medium (OCT). Cryostat (Leica CM-3050-S) sections (10 μm thick) were stored at −80° C. Organ sections were first permeabilized and blocked for 1 hr at room temperature using a solution of PBS 1×, 0.3% Triton X-100 and 10% of Normal Donkey Serum (NDS). Sections were then incubated overnight at 4° C. with anti-RAGE (rat, 1/500, R&D Systems #MAB1179), followed by anti-rat-Alexa 488 (donkey, 1/800, Jackson ImmunoResearch #712-545-153) for 2.30 hr at room temperature. Nuclei were stained with Hoechst (0.5 μg/mL, Life Technologies). Omission of the primary antibody was used as a control and no immunostaining was observed. Sections were mounted using Prolong Gold Antifading reagent (Life Technologies) on Superfrost glass slides. Images were taken and processed using an Apotome microscope (Zeiss, Jena, Germany) and Zen software (Zeiss).

These data demonstrate that RAGE is highly expressed in the lungs and, therefore, represents a valid target for delivery of therapeutic molecules.

### Example II

### Construction of CHO Cell Lines Stably Expressing Human and Mouse RAGE.

The prerequisite to the identification and characterization of RAGE-binding VHHs was the establishment of stable eukaryotic cell lines (Chinese hamster ovary cells, CHO) expressing human RAGE (hRAGE) and mouse RAGE (mRAGE), constitutively and at high rates. These cell lines were then used i) for the identification and characterization of agents binding to the receptor expressed at the cell surface, in its native configuration; and ii) to test whether the receptor could internalize such agents by endocytosis.

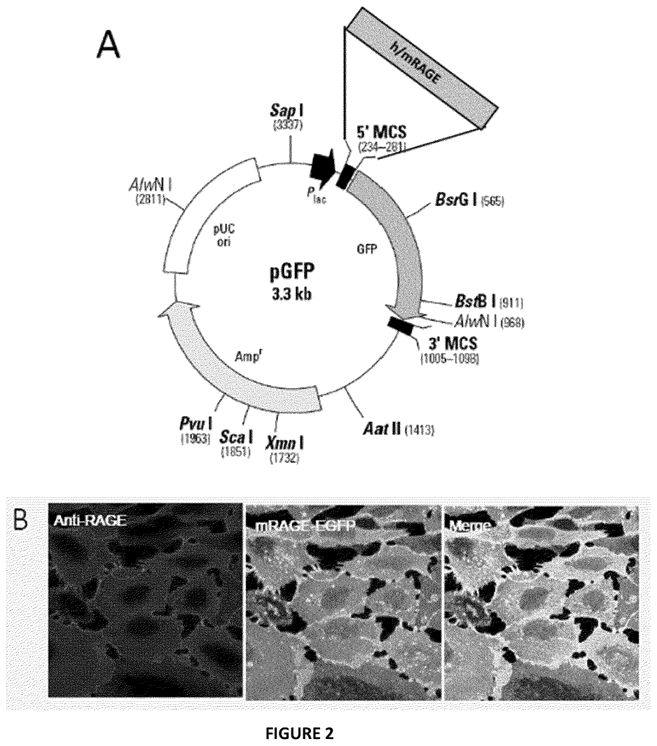

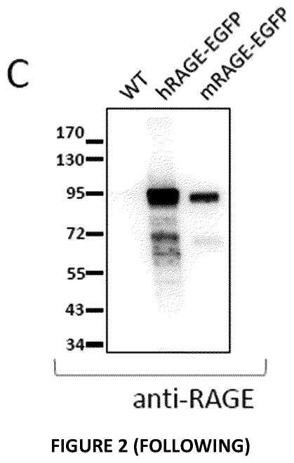

For the construction of these cell lines, the cDNAs coding for the hRAGE and mRAGE were cloned using sequence information available in databases (accession number: NM 001136.4 and NM_007425.3 respectively). The primers necessary for cDNA amplification by RT-PCR were selected (see table 1 below), comprising at their end (in bold type) the restriction sites (Sacl and SacII) necessary for cloning in the pEGFP-N1 expression vector (Clontech) (FIG. 2-A).

TABLE 1

| Receptor | |
|---|---|
| name | Primer sequences |
| hRAGE | (F) ATATATGAGCTCTGGAAGGAAGCAG |
| | GATGG (SEQ ID NO: 68) |
| | (R) TTAATTCCGCGGAGGCCCTCCAGTA |
| | CTACTCTCG (SEQ ID NO: 69) |
| mRAGE | (F) |
| | GGGATCCACCGGTCGCCACCATGCCAGCG |
| | GGGACAGCAG C (SEQ ID NO: 70) |
| | (R) |
| | TCCTCGCCCTTGCTCACCATCGGTCCCCC |
| | GGCACCAT TCTC (SEQ ID NO: 71) |

Total RNA prepared from human or mouse brain was used for RT-PCR amplification of the cDNA fragment coding for h/mRAGE. After amplification, the PCR product corresponding to hRAGE was digested by Sacl-SacII restriction enzymes and ligated in the pEGFP-N1 expression vector (Clontech), digested by the same restriction enzymes. mRAGE was introduced into pEGFP-N1 vector by mutagenesis. After transfection in eukaryotic cells, this vector enables the expression, under control of the CMV promoter, of the h/mRAGE fused to EGFP at its C-Terminal end, i.e., at the end of its intracellular domain. After transforming competent E. coli DH5α bacteria, obtaining isolated colonies and preparing plasmid DNA, both strands of the construct were fully sequenced for verification.

Transient transfections in CHO-K1 cells were carried out and used to select stable transfectants by limit dilution and resistance to antibiotic (G418). These cell lines were amplified while maintaining selective pressure.

Confocal photomicrographs taken after immunocytochemistry on fixed (PFA) cell lines using a rat anti-RAGE primary antibody (R&S Systems #MAB1179) diluted at 1/200 followed by an anti-rat A594-conjugated secondary antibody diluted at 1/800 confirm in FIG. 2-B, co-localization between EGFP (in green) and anti-RAGE antibody (in red) and therefore, good expression of the receptor, notably at the cellular membrane.

Membrane expression of the receptors of the expected size was checked by western blot on cell membranes of h/mRAGE-GFP CHO cell lines extracted with ProteoExtract Subcellular Proteome Extraction Kit. Proteins corresponding to the combined sizes of EGFP and h/mRAGE (95 kDa), were detected with the anti-RAGE antibody (FIG. 2-C). A CHO K1 wild type (WT) cell line was used as negative control and the anti-RAGE antibody detected no proteins.

These data confirm the expression of h/mRAGE receptor at the cell surface of the CHO cell lines that were generated. These cells can be used to immunize the llama to generate VHH that bind to RAGE.

### Example III

Generation of VHH that Bind RAGE

A llama (Lama glama) was immunized subcutaneously 4 times with membrane preparations from CHO stable cell lines expressing the human and murine receptor of interest. VHH library construction was performed as previously described (Alvarez-Rueda et al., 2007, Behar et al., 2009). Briefly, mRNAs coding for VHH were amplified by RT-PCR from the total RNAs of peripheral blood mononuclear cells isolated by ficoll gradient and cloned into the pHEN1 phagemid. Reiterative selections enabled the isolation of phages presenting VHH exhibiting strong affinity for the RAGE expressed at the cell surface.

In total, more than 700 clones were screened for their ability to bind RAGE, and approximately 70 clones were sequenced.

VHHs with improved binding (to both the murine and the human cell lines) and cell penetration were obtained. Illustrative VHH are VHH 1, VHH 2, VHH 3, VHH 4 and VHH 5 (see also the list of sequences). These VHHs do not bind to the control CHO cells.

The amino acid sequences of each of these VHH are provided in the Sequence Listing. Based on these studies, various VHH that bind to RAGE were generated and are included in the claimed invention.

### Example IV

### Determination of VHH-RAGE Binding Affinity.

The binding properties of VHHs with affinity for RAGE were tested using flow cytometry, and apparent affinities (K_{d app}) were determined. All experiments were performed in 96 well plates using 2×10⁵ cells/well, at 4° C. with shaking. CHO cell lines expressing RAGE fused to EGFP or CHO WT cells were saturated with PBS/BSA 2% solution during 30 min to avoid nonspecific binding, followed by incubation with purified VHHs at concentrations ranging from 50 μM to 0.5 nM for 1 hr. After one wash in PBS/BSA 2%, cells were incubated for 1 hr with an anti-6His tag antibody (mouse), washed twice with PBS/BSA 2%, and incubated for 1 hr with an Alexa647-conjugated anti-mouse secondary antibody. After two last washes in PBS/BSA 2%, cells were fixed by incubation for 15 min with PBS/PFA 2%, washed once with PBS and finally resuspended in PBS. Fluorescence levels were assessed using an Attune N×T flow cytometer (Thermo Fisher Scientific).

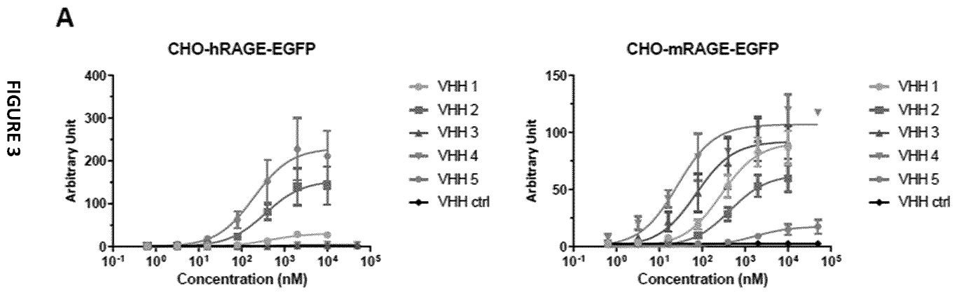

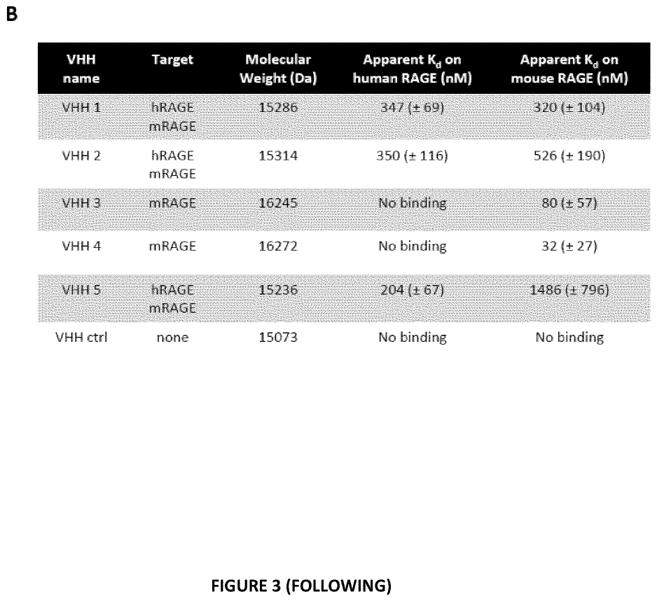

Three VHHs (VHH 1, 2 and 5) out of 5 bound both the human and the mouse RAGE and induced a concentration-dependent shift of the signal (FIG. 3-A). Two VHHs (VHH 3 and 4) bound only the mouse RAGE. There was no nonspecific labelling in the control conditions when cells were incubated with control VHH (VHH ctrl). Moreover, no labelling of the CHO WT control cells was detected with all the tested VHHs (not shown). The VHH K_{d app} were calculated using GraphPad Prism software. They ranged from 32 nM (VHH 4) to 1486 nM (VHH 5) on mRAGE, and from 204 nM (VHH 5) to 350 nM (VHH 2) on hRAGE (FIG. 3-B).

Based on these results, all the VHH display the required binding affinity (between 0.1 nM and 10 μM).

### Example V

Generation of VHH-siRNA Conjugates and Evaluation of their Binding Affinities

The conjugation strategy involved a convergent synthesis with the parallel modification of i) an amine-functionalized siRNA and ii) the VHH fused to a myc tag and a 6His tag. The amino-functionalized siRNAs, in this example an siTTRm (transthyretin) and an siSOD1m (Superoxide Dismutase 1), were chemically modified by conjugation of a heterobifunctional linker, which is, in the context of this particular example either DBCO-NHS or BCN-NHS, to introduce a cycloalkyne moiety (DBCO: dibenzocyclooctyne or BCN: bi-cyclooctyne). The VHHs were site-specifically modified using the BTG (Bacterial Transglutaminase) enzyme which catalyzes the formation of an isopeptidic bond between the glutamine residue (i.e., Q residue) in the myc tag sequence and an amino-functionalized substrate, to produce the azido-VHH intermediates.

In a final step, both the alkyne-siRNAs and the azido-VHHs were conjugated to each other by the copper-free click chemistry reaction.

The binding properties of VHH-siRNA conjugates with affinity for RAGE were tested using flow cytometry, and apparent affinities (K_{d app}) were determined. The same protocol as the one described in example IV was used.

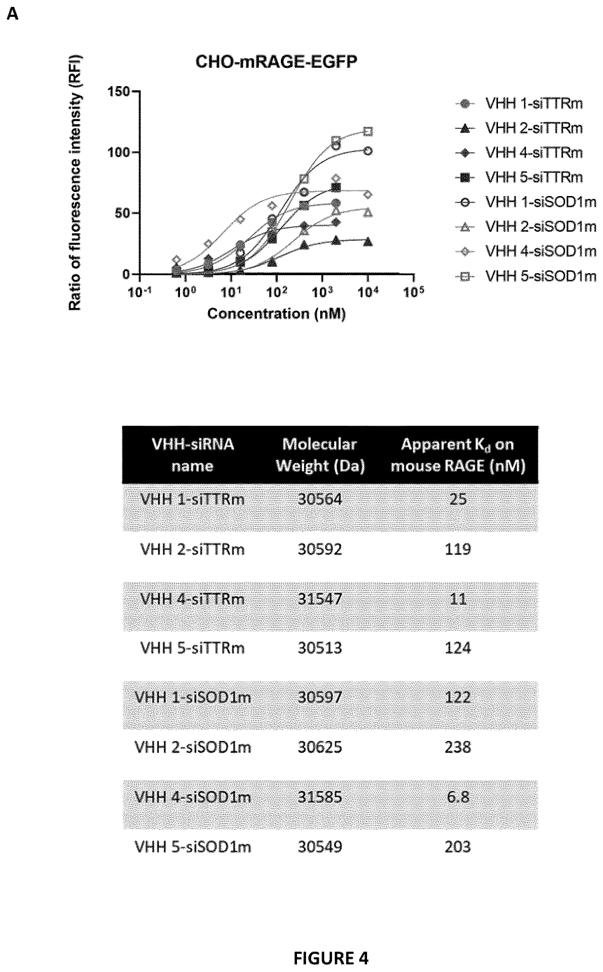

All VHH-siRNAs bound the mRAGE and induced a concentration-dependent shift of the signal (FIG. 4-A), confirming that the VHH conjugated to both the siTTRm and the siSOD1m retained the binding to the receptor of interest. The VHH-siRNA K_{d app} were calculated using GraphPad Prism software. They ranged from 6.8 nM (VHH 4-siSOD1m) to 238 nM (VHH 2-siSOD1m) (FIG. 4-B).

These data demonstrate that the VHH of the invention can be conjugated to therapeutic molecules such as siRNA and keep their affinity to RAGE.

### Example VI

Generation of VHH-RAGE-Fc Fusions and Evaluation of their Binding Affinities

Anti-RAGE VHH molecules of the invention were fused to an IgG Fc fragment. To produce the fusion protein, DNA fragments encoding the VHHs (with no tag) were amplified by PCR and cloned into the pINFUSE-IgG1-Fc2 vector (InvivoGen) to encode a human IgG1-Fc fragment encompassing in its N-ter or in its C-ter the VHHs. Fusion proteins were prepared using the Expi293 Expression System according to the manufacturer's instructions (Life Technologies). Seventy-two hrs post-transfection, supernatants were recovered and purified using Protein A GraviTrap columns (GE Healthcare). The purified fusion proteins were quantified at 280 nm using a Nanodrop apparatus (Thermo Fisher).

Immunocytochemistry experiments on CHO cell lines expressing the mouse or human RAGE fused to EGFP were performed to confirm the ability of fusion proteins to bind the RAGE. For these experiments, VHH-Fc fusion proteins were incubated at 50 nM on living cells, detected using an Alexa594-conjugated anti-hFc antibody after cells fixation with PBS-PFA 4% and permeabilization of the cell membrane with PBS-0.1% Triton X100 and photographed with a confocal microscope.

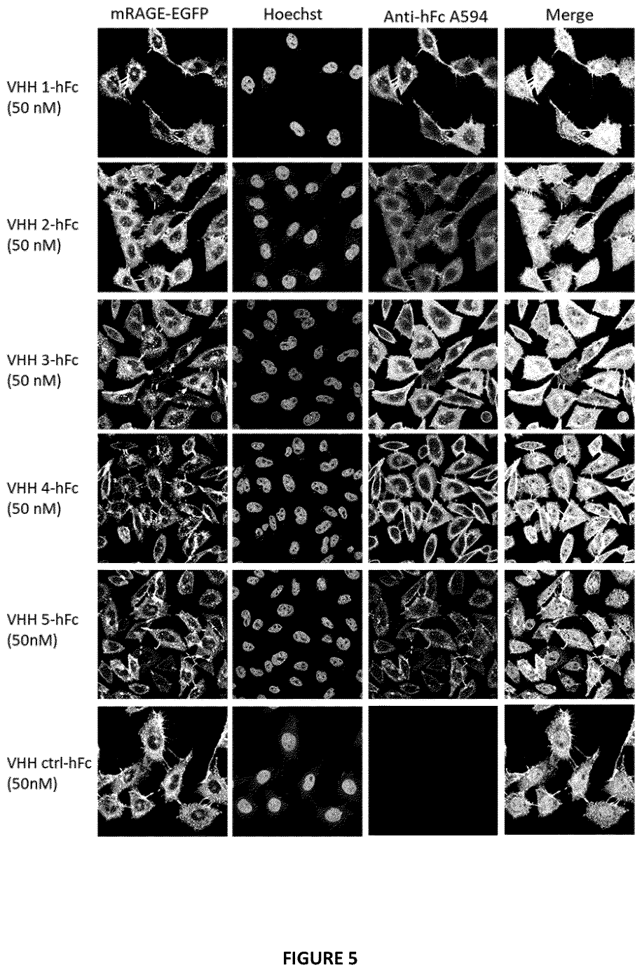

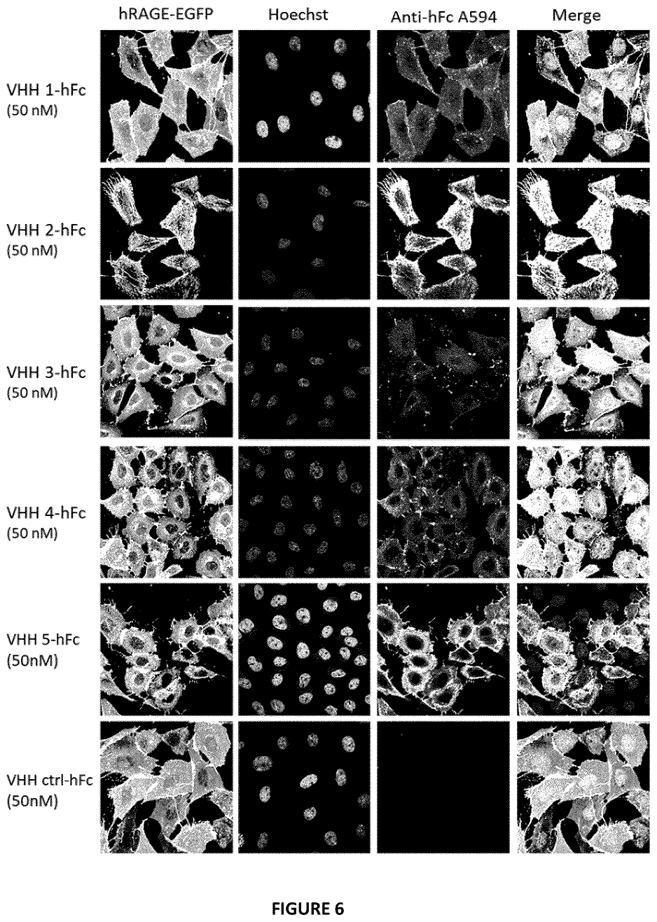

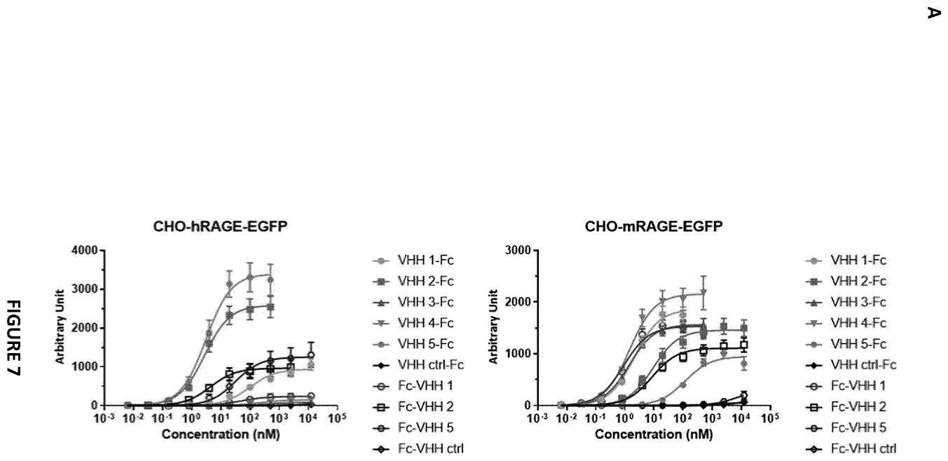

Results demonstrate the binding/uptake of VHHRAGE-Fc fusions of the invention to/by cells expressing the mRAGE or hRAGE (FIGS. 5 and 6). No binding of a control VHH-Fc conjugate (VHH ctrl-Fc) on cells was observed, showing the specificity of the interaction.

The binding properties of VHH-Fc and Fc-VHH fusion proteins with an affinity for the RAGE were tested in flow cytometry experiments, and apparent affinity (K_{d app}) were determined. All experiments were performed in 96 well plates using 2×10⁵ cells/well, at 4° C. with shaking. CHO cell lines expressing the receptors of interest fused to EGFP or CHO WT cells were saturated with PBS/BSA 2%, followed by an incubation with purified VHH-Fcs or Fc-VHHs at concentrations ranging from 12.5 μM to 6 μM for 1 hr. After 2 washes in PBS/BSA 2%, cells were incubated for 1 hr with an Alexa647-conjugated anti-hFc antibody. After two last washes in PBS/BSA 2%, cells were fixed by incubation for 15 min with PBS/PFA 2%, washed once with PBS and finally resuspended in PBS. Fluorescence levels were assessed using an Attune N×T flow cytometer (Thermo Fisher Scientific).

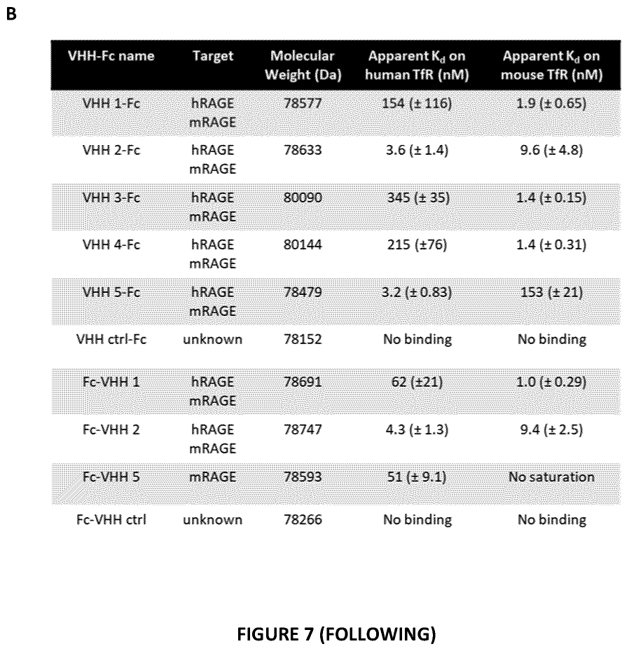

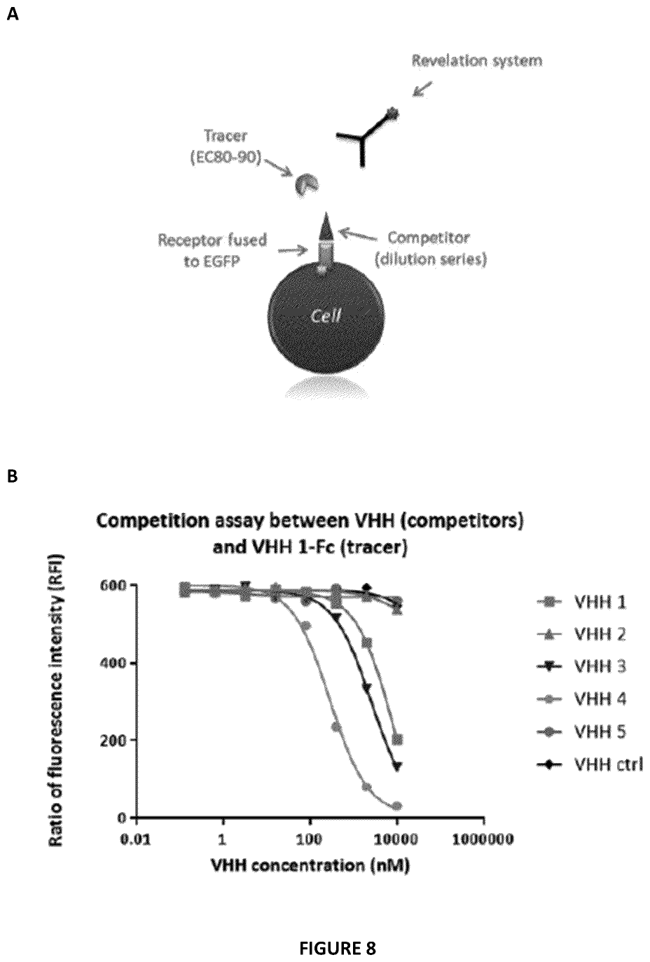

All VHH-Fc and Fc-VHH fusion proteins induced a concentration-dependent shift of the signal, confirming binding to the receptor of interest (FIG. 7-A). Noteworthy, VHH 3-Fc and VHH 4-Fc showed a binding to the human receptor. The VHH-Fc and Fc-VHH K_{d app} were calculated using GraphPad Prism software (FIG. 7-B). The K_{d app} of all VHHs were greatly improved by the conjugation with an Fc fragment, with K_{d app} ranging from 1 nM to 154 nM for RAGE-binding VHH-Fcs and Fc-VHHs. This is another example where inventors demonstrate that VHH of the invention can be conjugated to another therapeutic molecule (Fc fragment in this case) and keep their affinity to RAGE.

### Example VII

Competition Assay Between Purified VHHs with Affinity for the RAGE and VHH 1-Fc

To evaluate the ability of selected VHHs to compete with each other for the binding to the receptor, competition assays using flow cytometry experiments were performed. In a first step, competitors in dilution series were incubated on CHO cells expressing the receptor of interests fused to EGFP, for 1 hr at 4° C. Secondly, tracers at EC80 were added and incubated 1 hr more, and were then detected with the appropriate revelation system (FIG. 8-A).

Illustrative experiment showed that VHH 1-Fc, when used as tracer, was able to displace the binding of VHH 3 et VHH 4, suggesting that these three VHHs bind to the same or close epitope on mRAGE (FIG. 8-B).

### Example VIII

### Determination of VHH-RAGE-Fc Binding Domain on RAGE

The full-length isoform of RAGE extracellular domain encompasses three conserved domains composed of a V-type immunoglobulin (Ig) domain and two C-type Ig domains named C1 and C2.

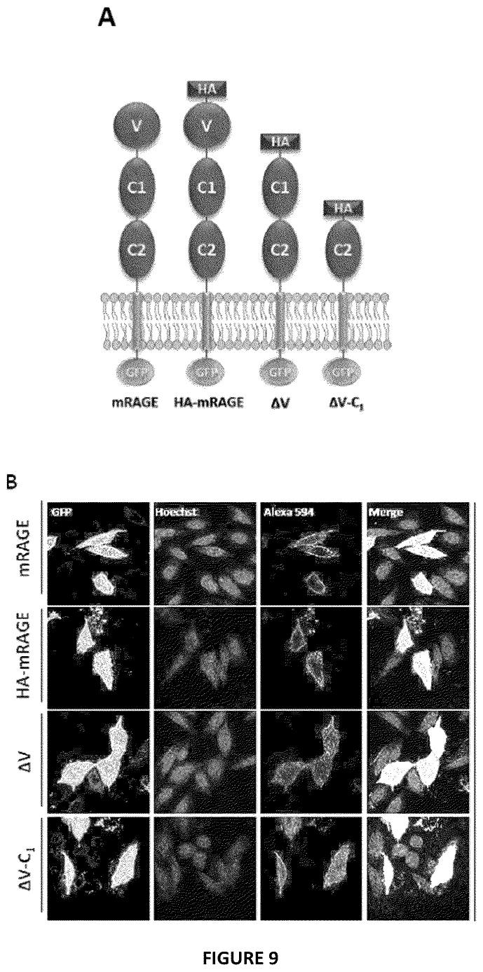

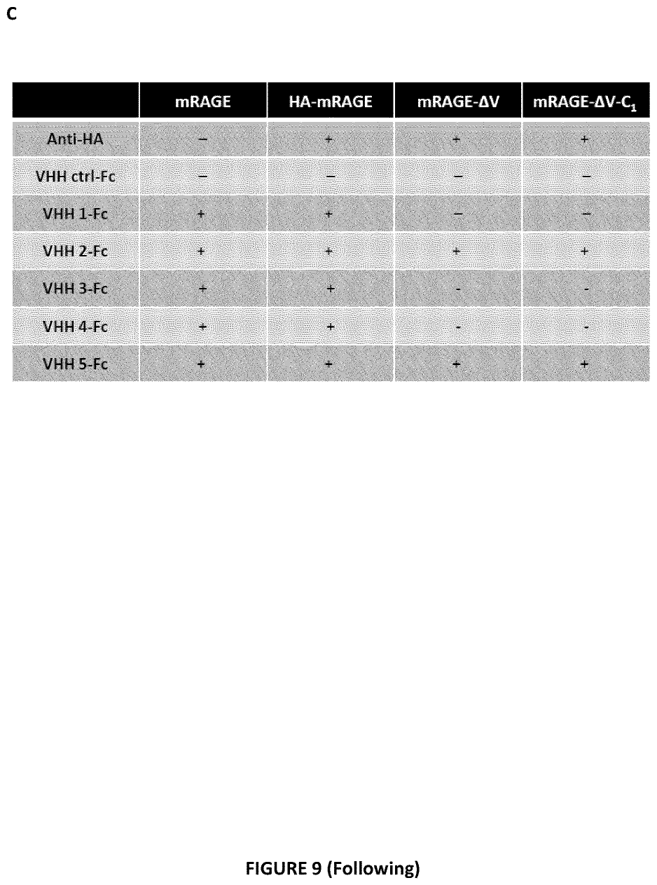

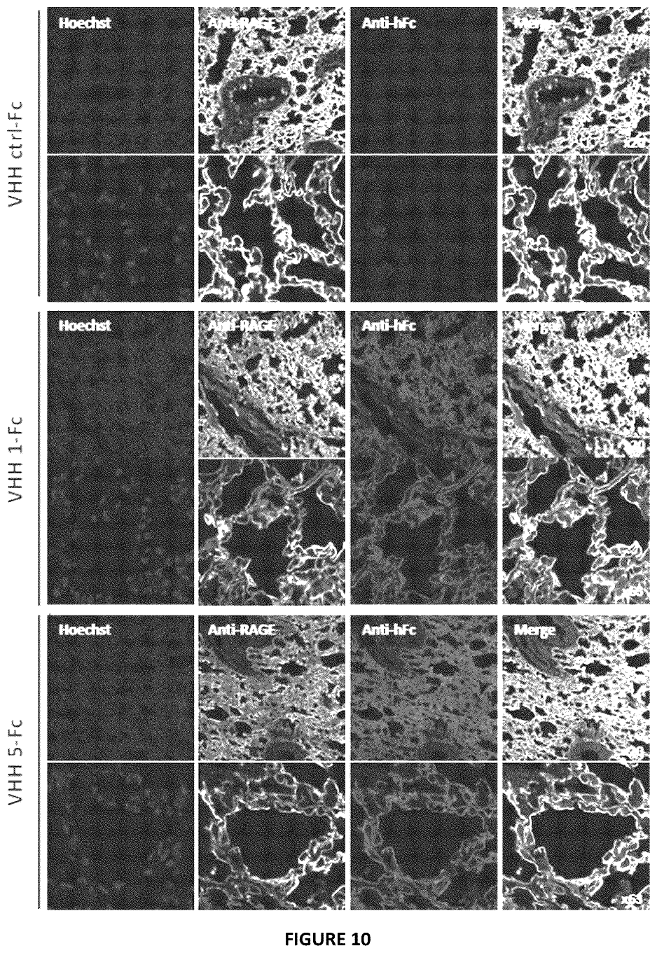

To determine the binding domain of VHH-RAGE-Fc, inventors suppressed RAGE domains sequentially, expressed the resulting truncated constructs mRAGE-ΔV, mRAGE-ΔV-C1 (FIG. 9-A) and compared VHHRAGE-Fc binding on the full-length construct (mRAGE and HA-mRAGE) in transfected CHO cells. An HA tag was introduced in the N-ter of each receptor to validate appropriate presentation of the extracellular domain using anti-HA, non-permeabilizing immunocytochemistry. EGFP was fused at the C-terminal end to enable easy visualization of the truncated and full-length receptor by fluorescence microscopy. Gene synthesis (GeneCust) was used to produce the DNA fragments coding amino-acids 1 to 380 of mature full-length mRAGE, amino acids 95 to 380 for mRAGE-ΔV and amino acids 204 to 380 for mRAGE-ΔV-C1. The synthetised DNA fragments were cloned into a pEGFP-N1 plasmid after restriction using XhoI EcoRI and ligation. CHO WT cells were transfected using jetPEI™ with the different plasmid constructs according to the manufacturer's instructions.

For immunocytochemistry, living transfected cells were incubated with 250 nM VHH-RAGE-hFc or anti-HA antibody for 1 hr at 37° C. Cells were then fixed using PBS-PFA 4%. VHHRAGE-hFc or anti-HA antibody were respectively revealed with mouse anti-human Fc Alexa 594 or donkey anti-rat Fc Alexa 594 and cells photographed with a confocal microscope.

Anti-HA immunocytochemistry (without cell permeabilization) indicated that the mRAGE-ΔV, mRAGE-ΔV-C1 and HA-mRAGE were correctly expressed at the plasma membrane. VHH 5-Fc, shown as an example (FIG. 9-B), binds full-length RAGE and all its truncated versions, meaning this VHH binds the C2 domain. The same binding profile was observed with VHH 2-Fc. On the opposite, VHH 1-Fc, VHH 3-Fc and VHH 4-Fc bind mRAGE and HA-mRAGE but not mRAGE-ΔV nor mRAGE-ΔV-C1, meaning that these VHH bind the V1 domain or VC1 domain. There was no binding of the irrelevant VHH ctrl-Fc on any of the constructs. The immunocytochemistry experiment results are summarized in FIG. 9-C.

These results indicate the binding domains of the different VHH:
VHH 1-Fc, VHH 3-Fc and VHH 4-Fc bind to V1 or VC1 domain
VHH 2-Fc and VHH 5-Fc bind to the C2 domain.

### Example IX

### Cellular Binding/Co-Localization of VHH-Fc on RAGE in Mice Lungs.

To confirm the ability of fusion proteins to bind RAGE in vivo, immunocytochemistry experiments were performed on lungs of mice injected into the tail vein at 35 nmol/kg with VHH ctrl-Fc, VHH 1-Fc and VHH 5-Fc. Mice were perfused with saline 48 hours post injection and lungs were incubated in PFA 4% overnight. Lungs were extensively washed in PBS 1× and incubated 2 days in sucrose 30% before being snap frozen. The fixed lung was embedded in OCT and cut in 14 μm thick sections. Double-immunofluorescent staining (IF) of lung tissue with anti-RAGE (primary antibody 1/200: R&D Mab1179 and secondary antibody 1/500 donkey anti-rat 488 JIR 712-545-153) and with VHH-Fc (1/100 goat anti-human Fc 594, JIR 109-605-098) antibodies were performed on lung sections. Cell nuclei were labeled with Hoechst #33342. Representative photographs were taken with a confocal microscope with a magnification of 20 and 63 times.

VHH-Fc administration does not alter the alveolar structure of the lung and as expected, a strong RAGE expression is observed at the plasma membrane of lung epithelial cells. In the merged pictures (far right panel), dual IF staining of RAGE and VHH-Fc shows co-localization between VHH 1-Fc or VHH 5-Fc and RAGE at the alveolar epithelium. No binding on RAGE is observed for the VHH ctrl-Fc (FIG. 10).

These results demonstrate that VHH 1-Fc and VHH 5-Fc target and bind RAGE in the lung in vivo whereas VHH ctrl-Fc failed to do so. No visible toxicity was observed in lungs 48 hrs post-administration.

These results demonstrate the significant accumulation of the RAGE-targeting conjugates (VHH 1-Fc and VHH 5-Fc) in the lungs.

### Example X

### Pharmacokinetic and Organ Uptake of VHH-Fc Conjugates In Vivo.

To assess the potential of VHH-Fc conjugates to target organs expressing RAGE receptor in vivo, conjugates VHH 1-Fc, VHH 4-Fc, VHH 5-Fc and VHH ctrl-Fc were injected intravenously into mice tail vein at the dose of 35 nmol/kg (n=4-12/time point). At different times post-injection (2 h, 6 h, 18 h, 48 h, 168 h) mice were deeply anesthetized with mixture of ketamine (100 mg/kg) and xylazine (10 mg/kg) administered by intra-peritoneal route. Blood was collected, within Na heparin tube, by cardiac puncture directly from the right ventricle. Plasma was recovered after blood centrifugation at 1500 g for 10 min and then stored at −80° C. until analysis. Mice were then extensively perfused in the left ventricle to remove any blood traces in the organs with heparinized 0.9% NaCl solution. After perfusion, different organs (lungs, kidney and liver) were sampled, weighed, homogenized in lysis buffer (PBS triton 0.1% with anti-protease) and transferred into Safe-Lock Eppendorf tubes, snap frozen and stored at −80° C. until bioanalysis.

The amount of VHH-Fc in plasma and lysed organs was measured using an in house anti-Fc ELISA. Results are presented as concentrations (nM), percentage of injected dose per gram tissue, or by organ-to-plasma ratio (FIG. 11).

RAGE-binding fusion proteins VHH 1-Fc, VHH 4-Fc and VHH 5-Fc and VHH ctrl-Fc show different pharmacokinetic profiles (FIG. 11-A, C). VHH 1-Fc is rapidly distributed with 19% ID measurable in plasma 2 hrs post-injection (pi). The distribution phases last approximately 6 hrs for VHH 1-Fc, VHH 5-Fc and VHH ctrl-Fc with 19%, 41% and 34% ID still present in plasma, respectively at this time (FIG. 11-B, D). At 48 h post injection, the elimination phase of all molecules is similar. Plasma pharmacokinetic parameters of all VHH-Fc, analyzed using the Kinetica software, are shown in tables 2A, 2B, 2C and 2D below.

A Plasma

| | Cmax | % | | | | | | |
|---|---|---|---|---|---|---|---|---|
| | (nmol/L | IDmax/g | T_{1/2} | AUC_{0-168 h} | AUC_{0-inf} | Clearance | Vd | MRT |
| Molecule | or /Kg) | % | (h) | (nmol/L/h) | (nmol/L/h) | (mL/d/Kg) | (mL) | (h) |
| VHH ctrl-Fc | 475.1 | 56.5 | 187.8 | 26986.1 | 54808.3 | 15.3 | 4.3 | 256.3 |
| VHH 1-Fc | 439.1 | 52.4 | 108.2 | 13207.5 | 20101.4 | 41.5 | 6.7 | 295.3 |
| VHH 4-Fc | 157.8 | 17.4 | 208.3 | 18341.6 | 41758.8 | 20.6 | 6.4 | 114.9 |
| VHH 5-Fc | 455.6 | 54.6 | 80.4 | 29902.9 | 39132.8 | 21.2 | 2.6 | 153.0 |

B Lung

| | | Cmax | % | | | |
|---|---|---|---|---|---|---|
| | Tmax | (nmol/L | IDmax/g | T_{1/2} | AUC_{0-168 h} | AUC_{0-inf} |
| Molecule | (h) | or /Kg) | % | (h) | (nmol/L/h) | (nmol/L/h) |
| VHH | 6 | 15.5 | 1.6 | 196.6 | 1674.0 | 3667.3 |
| ctrl-Fc | | | | | | |
| VHH 1-Fc | 96 | 79.5 | 9.5 | — | 10372.4 | — |
| VHH 4-Fc | 48 | 77.5 | 9.5 | 90.7 | 8669.3 | 12960.9 |
| VHH 5-Fc | 48 | 192.5 | 22.9 | 63.6 | 19043.7 | 23890.3 |

C Liver

| | | Cmax | % | | | |
|---|---|---|---|---|---|---|
| | Tmax | (nmol/L | IDmax/g | T_{1/2} | AUC_{0-168 h} | AUC_{0-inf} |
| Molecule | (h) | or /Kg) | % | (h) | (nmol/L/h) | (nmol/L/h) |
| VHH | 6 | 11.8 | 1.5 | 251.5 | 1364.3 | 3737.7 |
| ctrl-Fc | | | | | | |
| VHH 1-Fc | 6 | 16.3 | 1.7 | 90.9 | 1218.0 | 1691.8 |
| VHH 4-Fc | 6 | 14.8 | 1.7 | 121.7 | 1931.1 | 3200.4 |
| VHH 5-Fc | 6 | 15.1 | 1.8 | 213.2 | 1822.5 | 4426.8 |

D Kidney

| | | Cmax | % | | | |
|---|---|---|---|---|---|---|
| | Tmax | (nmol/L | IDmax/g | T_{1/2} | AUC_{0-168 h} | AUC_{0-inf} |
| Molecule | (h) | or /Kg) | % | (h) | (nmol/L/h) | (nmol/L/h) |
| VHH | 6 | 15.6 | 1.6 | 271.9 | 1747.4 | 5007.2 |
| ctrl-Fc | | | | | | |
| VHH 1-Fc | 6 | 20.3 | 2.2 | 174.9 | 1527.8 | 3052.7 |
| VHH 4-Fc | 6 | 11.3 | 1.3 | 285.9 | 1569.3 | 4634.5 |
| VHH 5-Fc | 6 | 11.4 | 1.4 | 206.4 | 1600.0 | 3818.7 |

Table 2: Pharmacokinetic parameters of VHH-Fc fusion injected in WT C57Bl/6 mice. VHH ctrl-Fc, VHH 1-Fc, VHH 4-Fc and VHH 5-Fc were injected into tail vein at 35 nmol/kg and the mice were perfused with saline at 2, 6, 18, 48, 96 or 168 hours post injection. Amounts of VHH-Fc in plasma (A), lungs (B), liver (C) and kidney (D) were assessed by ELISA and the pharmacokinetic parameters were calculated using the Kinetica software.

A strong targeting to lungs is observed with all VHH-Fc from 18 hrs post-injection (pi) up to 168 hrs pi. The peak concentration is obtained 48 hrs post-injection with a concentration of 59.5 nM (6.9% ID) for VHH 1-Fc, 77.5 nM (9.5% ID) for VHH 4-Fc and 192.5 nM (22.9% ID) for VHH 5-Fc (FIG. 11-E, F). There was no significant accumulation of the control VHH ctrl-Fc (10 nM; 1% ID) (FIG. 11-E, F). The same distribution advantages are observed when evaluating lung-to-plasma ratios (FIG. 11-G). VHH 1-Fc, VHH 4-Fc and VHH 5-Fc lung targeting is confirmed, with a strong accumulation that lasted up to 168 hrs.

In the liver, all VHH-Fc show the same profile with no significant accumulation compared to VHH ctrl-Fc. (FIG. 11-H, I). In kidney, 2 hrs pi, VHH 1-Fc accumulate 1.6 times more compared to VHH ctrl-Fc. VHH 4-Fc and VHH 5-Fc are identical to VHH ctrl-Fc (FIG. 11-K, L). Significant advantage is observed for VHH 1-Fc compared to the control when evaluating liver-to-plasma and kidney-to-plasma ratios, at every time points except 168 hrs (FIG. 11-J, M).

The pharmacokinetic parameters in the lung, liver and kidney, were estimated by non-compartmental analysis. An important uptake is found for the lung for all VHH-Fc compared to VHH ctrl-Fc, with VHH 5-Fc having the highest values of Cmax, AUC_{0-168 h} and % IDmax (Table 1 B). By contrast, VHH 1-Fc, VHH 4-Fc, VHH 5-Fc and VHH ctrl-Fc have comparable Cmax, AUC_{0-168 h} and % IDmax for the liver, and kidney (Table 1 C, D).

These results demonstrate that RAGE-targeting VHHs of the invention can be used to effectively deliver or improve the biodistribution in the lungs of therapeutic agents. VHH 1-Fc, VHH 2-Fc, VHH 3-Fc, VHH 4-Fc and VHH 5-Fc, in particular VHH 1-Fc, VHH 4-Fc and VHH 5-Fc, show an advantageous preferential lung targeting compared to other organs that express low levels of RAGE (e.g., liver and kidneys).

### Example XI

VHH-RAGE LNP Characterization, In Vitro mLuc mRNA Delivery to h/mRAGE-GFP CHO Cells and In Vivo Distribution in C57/B16 Mice at 6 hr.

To confirm the capacity of VHH-RAGE to deliver specifically desired cargo, we functionalized lipid nanoparticles (LNPs) on their surface. These LNPs were loaded with Fluc luciferase mRNA to measure their efficiency. The prepared vectorized LNPs were compared to non-vectorized LNPs (naked LNPs) and LNPs functionalized with irrelevant VHH (VHH ctrl). The conjugation of the vector to the LNPs was achieved through click chemistry, specifically the SPAAC reaction between a reactive moiety, BCN (bicyclo[6.1.0]nonyne), selectively conjugated to the VHH-RAGE and a corresponding functional group introduced via an additional lipid ingredient (phospholipid-azide; DOPE-PEG2OOO-N3) used during LNP preparation. The resulting LNPs were purified through multiple filtration steps using a 100 kDa MWCO (Amicon, Sigma-Aldrich) membrane and characterized using dynamic light scattering (DLS), a cholesterol standard assay kit, and the Quant-iT™ RiboGreen RNA Assay to determine mRNA concentration and encapsulation efficiency.

The hydrodynamic diameter and polydispersity index (PDI) of the LNPs were determined through dynamic light scattering (DLS). Measurements were performed on samples diluted tenfold in Dulbecco's phosphate-buffered saline (DPBS) using the Zetasizer Nano Series Advanced Blue instrument (Malvern Instruments, Malvern Panalytical, Malvern, UK). Each sample underwent three runs, each lasting 5 minutes, at a temperature of 25° C. with a 173° backscatter setup, and the results were averaged.

The mRNA concentration was determined using the Quant-iT RiboGreen mRNA Broad Range Assay Kit following the standard protocol. LNPs were incubated with 0.5% (v/v) Triton X-100 (for total mRNA concentration) or with DPBS (for free mRNA) at RT for 5 minutes. Encapsulation efficiency was calculated using the formula [(total mRNA concentration−free mRNA)/total mRNA concentration]×100.

Cholesterol content was measured using an enzymatic spectrophotometric assay (MAK043, Sigma-Aldrich) following the standard procedure, with an average of two samples (diluted tenfold in Dulbecco's phosphate-buffered saline (DPBS)).

The ability of VHHs-LNP to deliver FLuc mRNA into cells was investigated on CHO-hRAGE-EGFP and CHO-mRAGE-EGFP cell lines. Naked LNP, VHH ctrl-LNP and VHH 5-LNP were diluted at 1.25 μg mRNA luc/ml in OptiMEM medium (Thermo Fisher scientific) and incubated on the cells at 37° C. for 6 hours (2×105 cells/well). At the end of the incubation time, ONE-Glo™ Luciferase Assay System (Promega) containing 5′-Fluoroluciferin substrate was added on the cells (in a one-to-one ratio with culture medium) for 15 minutes to enable complete cell lysis. The mix was transferred into 96 wells white plate and luminescence produced by oxygenation of 5′-Fluoroluciferin by luciferase protein was read by a Glomax navigator (with an integration time 0.3 sec).

Luminescence values were significantly higher for CHO-hRAGE-EGFP and CHO-mRAGE-EGFP treated with VHH 5-LNP than VHH ctrl-LNP whereas low and similar luminescence were measured between the two control LNP: naked LNP and VHH ctrl-LNP.

These results demonstrate that the functionalization of LNP with VHH 5 enables to enhance by 3 to 5-fold the cellular delivery of FLuc mRNA via a RAGE-dependent mechanism.

To assess the potential of VHH-LNP to target the lung (that have a basal high expression level of RAGE) in vivo, VHH 5-LNP and VHH ctrl-LNP were injected intravenously into mice tail vein at the dose of 20 μg/200 μL of mLuc mRNA per mice (n=3/4 mice per VHH-LNP construct). Six hours post-injection mice were deeply anesthetized with mixture of ketamine (100 mg/kg) and xylazine (10 mg/kg) administered by intra-peritoneal route. Different organs (lung, gastrocnemius muscle, heart, kidney and brain) were sampled, weighed and ground in Precellys tubes (Bertin Technologies) with passive lysis buffer 1× (2 μl/mg tissue) from Promega. Lysed tissues were centrifuged at 13500 rpm at 4° C. for 10 min and supernatant was transferred into Safe-Lock Eppendorf tubes, snap frozen and stored at −80° C. until bioanalysis.

FLuc mRNA delivery by VHHs-LNP and tissue distribution was assessed by quantifying the levels of translated luciferase luminescence in each organ. In a 96 wells white plate, 20 μl of tissues lysate was mixed with 100 μl of ONE-Glo™ Luciferase Assay System. After 3 minutes of incubation, luminescence was read by a Glomax navigator (with an integration time of 0.3 sec).

A strong luminescence signal (RLU) was measured in the lungs of mice injected with VHH 5-LNP. Interestingly, this luminescence signal was 9-fold significantly higher than the one measured for the mice injected with VHH ctrl-LNP. Moreover, the luminescence values measured for VHH 5-LNP in kidney, muscle, heart, and brain tissue were 6 to 22-fold lower than the one measured in lung, demonstrating that VHH 5-LNP shows an advantageous preferential lung targeting compared to other organs that express low levels of RAGE.

Overall, these results demonstrate that RAGE-targeting VHHs of the invention can be used to effectively deliver or improve the distribution in/to the lungs of therapeutic agents, such as mRNA/LNP particles.

### LIST OF SEQUENCES

TABLE 3

| sdAb | Amino acid sequence | CDR1 | CDR2 | CDR3 |
|---|---|---|---|---|
| VHH 1 | QVQLVQSGGGLVQPGGSLRLSCAVSGSIFS | GSIFS | ISSGG | RLQANFG |
| | SATMGWYRQAPGKQRELAARISSGGSTNY | SAT | ST | LVDY |
| | LDSVKGRFTISRDNAKNTVYLQMNSLKLE | SEQ 1 | SEQ 2 | SEQ 3 |
| | DTAVYYCRLQANFGLVDYWGKGTQVTVS | | | |
| | S(AAAEQKLISEEDLNGAAHHHHHHGS)ₓ, | | | |
| | with x = 0 or 1 | | | |
| | SEQ 4 (x = 0) or SEQ 31 (x = 1) | | | |
| VHH 2 | QVQLVQSGGGLVQAGGSLRLSCAASGSVS | GSVSS | ITGRG | KIDRRGY |
| | SIYAMGWYRQAPGKQREWVTTITGRGITD | IYA | IT | APY |
| | YAASVKGRFTISRDTAKNAVYLQMNNLKP | SEQ 5 | SEQ 6 | SEQ 7 |
| | EDTARYYCKIDRRGYAPYWGQGTQVTVSS | | | |
| | (AAAEQKLISEEDLNGAAHHHHHHGS)ₓ, | | | |
| | with x = 0 or 1 | | | |
| | SEQ 8 (x = 0) or SEQ 32 (x = 1) | | | |
| VHH 3 | QVQLVQSGGGLVQAGGSLRLSCVASGRTF | GRTFS | ISWSG | ATEAGAF |
| | SNYAMAWFRQAPGEEREFVARISWSGGRT | NYA | GRT | GGVYSST |
| | SYADSVKGRFTISRDNAKNTVYLQMISLKP | SEQ 9 | SEQ 10 | GSYDY |
| | EDTAVYYCATEAGAFGGVYSSTGSYDYW | | | SEQ 11 |
| | GQGTQVTVSS(AAAEQKLISEEDLNGAAHH | | | |
| | HHHHGS)ₓ, with x = 0 or 1 | | | |
| | SEQ 12 (x = 0) or SEQ 33 (x = 1) | | | |
| VHH 4 | EVQLVESGGGLVQAGGSLRLSCVASGRTFS | GRTFS | ISWRG | ASEGGAF |
| | NYAMAWFRQAPGEEREFVARISWRGGRTS | NYA | GRT | GGVYSAT |
| | YADSVKGRFTISRDNAKNTVYLQMISLKPE | SEQ 13 | SEQ 14 | GSYDY |
| | DTAVYYCASEGGAFGGVYSATGSYDYWG | | | SEQ 15 |
| | QGTQVTVSS(AAAEQKLISEEDLNGAAHHH | | | |
| | HHHGS)ₓ, with x = 0 or 1 | | | |
| | SEQ 16 (x = 0) or SEQ 34 (x = 1) | | | |
| VHH 5 | EVQLVESGGGLVQPGGSLRLSCAASGTIFS | GTIFS | ISDSG | RSTFAAG |
| | LNPMGWYRQAPGKQLELVALISDSGTANY | LNP | TA | NFY |
| | ADFVRGRFTISRDTTKKTLYLQMNSLEPED | SEQ 17 | SEQ 18 | SEQ 19 |
| | TAVYYCRSTFAAGNFYWGQGTQVTVSS(A | | | |
| | AAEQKLISEEDLNGAAHHHHHHGS)ₓ, | | | |
| | with x = 0 or 1 | | | |
| | SEQ 20 (x = 0) or SEQ 35 (x = 1) | | | |

TABLE 4

| sdAb | SEQ ID | nucleotide sequences (including optional tag sequences) |
|---|---|---|
| VHH 1 | 21 (x = 0) | CAGGTGCAGCTGGTGCAGTCTGGGGGAGGCTTGGTGCAGCCTGGGGGGTCTCTGAG |
| | or 22 (x = 1) | ACTCTCCTGTGCAGTCTCTGGAAGTATCTTCAGTAGTGCTACCATGGGCTGGTACC |
| | | GCCAGGCTCCGGGGAAGCAGCGCGAGTTGGCCGCACGTATTTCTAGTGGTGGTAGC |
| | | ACAAACTATTTAGACTCCGTGAAGGGCCGATTCACCATCTCCAGAGACAACGCCAA |
| | | GAACACGGTGTATCTACAAATGAACAGTCTGAAACTTGAGGACACGGCCGTCTATT |
| | | ACTGTAGGTTGCAGGCTAATTTTGGCCTCGTGGACTATTGGGGCAAAGGGACCCAG |
| | | GTCACCGTCTCCTCA(GCGGCCGCAGAACAAAAACTCATCTCAGAAGAGGATCTGA |
| | | ATGGGGCCGCACATCACCACCATCACCATGGGAGCTAG)x, |
| | | with x = 0 or 1 |
| VHH 2 | 23 (x = 0) | CAGGTGCAGCTGGTGCAGTCTGGGGGAGGCTTGGTGCAGGCTGGGGGGTCTCTGAG |
| | or 24 (x = 1) | ACTCTCCTGTGCAGCCTCTGGAAGCGTCTCCAGTATTTATGCCATGGGCTGGTACC |
| | | GGCAGGCTCCCGGGAAGCAGCGCGAGTGGGTCACAACTATCACTGGTCGTGGTATT |
| | | ACCGACTATGCAGCCTCCGTGAAGGGCCGGTTCACCATCTCCAGAGACACCGCCAA |
| | | GAACGCGGTATATCTCCAAATGAACAACCTGAAACCTGAGGACACGGCCCGCTATT |
| | | ACTGCAAGATAGATCGTAGGGGCTATGCGCCCTACTGGGGCCAGGGGACCCAGGTC |
| | | ACTGTCTCCTCA(GCGGCCGCAGAACAAAAACTCATCTCAGAAGAGGATCTGAATG |
| | | GGGCCGCACATCACCACCATCACCATGGGAGCTAG)x, with x = 0 or 1 |
| VHH 3 | 25 (x = 0) | CAGGTGCAGCTGGTGCAGTCTGGGGGAGGATTGGTGCAGGCTGGGGGCTCTCTGAG |
| | or 26 (x = 1) | ACTCTCCTGTGTAGCCTCTGGACGCACCTTCAGTAACTATGCCATGGCCTGGTTCC |
| | | GCCAGGCTCCAGGCGAGGAGCGTGAGTTTGTAGCACGTATTAGTTGGAGCGGTGGT |
| | | AGGACATCATATGCAGACTCCGTGAAGGGCCGATTCACCATCTCCAGAGACAACGC |
| | | CAAGAACACGGTGTATCTGCAAATGATCAGCCTGAAACCTGAGGACACGGCCGTTT |
| | | ATTACTGTGCAACAGAGGCCGGCGCTTTTGGTGGTGTATACTCCTCTACAGGGTCT |
| | | TATGACTACTGGGGCCAGGGGACCCAGGTCACTGTCTCCTCA(GCGGCCGCAGAAC |
| | | AAAAACTCATCTCAGAAGAGGATCTGAATGGGGCCGCACATCACCACCATCACCAT |
| | | GGGAGCTAG)x, with x = 0 or 1 |
| VHH 4 | 27 (x = 0) | GAGGTGCAGCTGGTGGAGTCTGGGGGAGGATTGGTGCAGGCTGGGGGCTCTCTGAG |
| | or 28 (x = 1) | ACTCTCCTGTGTAGCCTCTGGACGCACCTTCAGTAACTATGCCATGGCCTGGTTCC |
| | | GCCAGGCTCCAGGCGAGGAGCGTGAGTTTGTAGCACGTATTAGTTGGCGCGGTGGT |
| | | AGGACATCATATGCAGACTCCGTGAAGGGCCGATTCACCATCTCCAGAGACAACGC |
| | | CAAGAACACGGTGTATCTGCAAATGATCAGCCTGAAACCTGAGGACACGGCCGTTT |
| | | ATTACTGTGCATCAGAGGGCGGCGCTTTTGGTGGTGTGTACTCCGCTACAGGGTCT |
| | | TATGACTACTGGGGCCAGGGGACCCAGGTCACTGTCTCCTCA(GCGGCCGCAGAAC |
| | | AAAAACTCATCTCAGAAGAGGATCTGAATGGGGCCGCACATCACCACCATCACCAT |
| | | GGGAGCTAG)x, with x = 0 or 1 |
| VHH 5 | 29 (x = 0) | GAGGTGCAGCTGGTGGAGTCTGGGGGAGGCTTGGTGCAGCCTGGGGGGTCTCTGAG |
| | or 30 (x = 1) | ACTCTCCTGTGCAGCCTCTGGAACCATCTTCAGTCTCAATCCCATGGGCTGGTACC |
| | | GCCAGGCTCCAGGGAAACAGCTCGAGTTGGTCGCACTAATTTCGGATAGTGGAACC |
| | | GCAAACTATGCAGACTTCGTGAGGGGCCGATTCACCATCTCCAGAGACACCACCAA |
| | | GAAAACGCTATATCTGCAGATGAACAGCCTTGAACCTGAGGACACAGCCGTCTATT |
| | | ACTGTAGATCTACATTCGCGGCGGGGAATTTTTACTGGGGCCAGGGGACCCAGGTC |
| | | ACTGTCTCCTCA(GCGGCCGCAGAACAAAAACTCATCTCAGAAGAGGATCTGAATG |
| | | GGGCCGCACATCACCACCATCACCATGGGAGCTAG)x, with x = 0 or 1 |

### REFERENCES

Bierhaus, A., S. Schiekofer, M. Schwaninger, M. Andrassy, P. M. Humpert, J. Chen, M. Hong, et al. «Diabetes-Associated Sustained Activation of the Transcription Factor Nuclear Factor-KappaB». Diabetes 50, no 12 (décembre 2001): 2792-2808. https://doi.org/10.2337/diabetes.50.12.2792.
Bongarzone, S., Savickas, V., Luzi, F., and Gee, A. D. (2017) Targeting the Receptor for Advanced Glycation Endproducts (RAGE): A Medicinal Chemistry Perspective. Journal of medicinal chemistry 60, 7213-7232.
Brett, J., A. M. Schmidt, S. D. Yan, Y. S. Zou, E. Weidman, D. Pinsky, R. Nowygrod, M. Neeper, C. Przysiecki, A. Shaw and et al. (1993). “Survey of the distribution of a newly characterized receptor for advanced glycation end products in tissues.” Am J Pathol 143(6): 1699-1712.
Chavakis, T., Bierhaus, A., and Nawroth, P. P. (2004) RAGE (receptor for advanced glycation end products): a central player in the inflammatory response. Microbes and infection 6, 1219-1225.
Dong, Hanbing, Yue Zhang, Yu Huang, et Hui Deng. «Pathophysiology of RAGE in Inflammatory Diseases». Frontiers in Immunology 13 (2022): 931473. https://doi.org/10.3389/fimmu.2022.931473.
He, S., Gui, J., Xiong, K. et al. A roadmap to pulmonary delivery strategies for the treatment of infectious lung diseases. J Nanobiotechnol 20, 101, 2022.
Healey, G. D., Pan-Castillo, B., Garcia-Parra, J., Davies, J., Roberts, S., Jones, E., Dhar, K., Nandanan, S., Tofazzal, N., Piggott, L., Clarkson, R., Seaton, G., Frostell, A., Fagge, T., McKee, C., Margarit, L., Conlan, R. S., and Gonzalez, D. (2019) Antibody drug conjugates against the receptor for advanced glycation end products (RAGE), a novel therapeutic target in endometrial cancer. J Immunother Cancer 7, 280
Hudson, Barry I., Angela M. Carter, Evis Harja, Anastasia Z. Kalea, Maria Arriero, Hojin Yang, Peter J. Grant, et Ann Marie Schmidt. «Identification, Classification, and Expression of RAGE Gene Splice Variants». FASEB Journal: Official Publication of the Federation of American Societies for Experimental Biology 22, no 5 (mai 2008): 1572-80. https://doi.org/10.1096/fj.07-9909com.
Khaket, T. P., Kang, S. C., and Mukherjee, T. K. (2019) The Potential of Receptor for Advanced Glycation End Products (RAGE) as a Therapeutic Target for Lung Associated Diseases. Curr Drug Targets 20, 679-689
Leclerc, Estelle, Günter Fritz, Mirjam Weibel, Claus W. Heizmann, et Arnaud Galichet. «S100B and S100A6 Differentially Modulate Cell Survival by Interacting with Distinct RAGE (Receptor for Advanced Glycation End Products) Immunoglobulin Domains». The Journal of Biological Chemistry 282, no 43 (26 Oct. 2007): 31317-31. https://doi.org/10.1074/jbc.M703951200.
Lee, Eun Ji, et Jong Hoon Park. «Receptor for Advanced Glycation Endproducts (RAGE), Its Ligands, and Soluble RAGE: Potential Biomarkers for Diagnosis and Therapeutic Targets for Human Renal Diseases». Genomics & Informatics 11, no 4 (décembre 2013): 224-29. https://doi.org/10.5808/GI.2013.11.4.224.
Majumdar S. and Siahaan T J., “Peptide-mediated targeted drug delivery”. Med Res Rev., 2012 May; 32(3):637-58.
Mohammed, A., Zeng, W., Mengist, H. M., Kombe Kombe, A. J., Ou, H., Yang, Y., Dan, Z., Xu, Z., Ma, H., and Jin, T. (2021) Generation, biochemical characterizations and validation of potent nanobodies derived from alpaca specific for human receptor of advanced glycation end product. Biochemical and biophysical research communications 581, 38-45
Neeper, Schmidt, et al, Cloning and expression of a cell surface receptor for advanced glycosylation end products of proteins. J Biol Chem 1992 Jul. 25; 267(21):14998-5004.
Ostendorp, Thorsten, Estelle Leclerc, Arnaud Galichet, Michael Koch, Nina Demling, Bernd Weigle, Claus W. Heizmann, Peter M. H. Kroneck, et Günter Fritz. «Structural and Functional Insights into RAGE Activation by Multimeric S100B». The EMBO Journal 26, no 16 (22 Aug. 2007): 3868-78. https://doi.org/10.1038/sj.emboj.7601805.
Raucci, Angela, Simona Cugusi, Antonella Antonelli, Silvia M. Barabino, Lucilla Monti, Angelika Bierhaus, Karina Reiss, Paul Saftig, et Marco E. Bianchi. «A Soluble Form of the Receptor for Advanced Glycation Endproducts (RAGE) Is Produced by Proteolytic Cleavage of the Membrane-Bound Form by the Sheddase a Disintegrin and Metalloprotease 10 (ADAM10)». FASEB Journal: Official Publication of the Federation of American Societies for Experimental Biology 22, no 10 (octobre 2008): 3716-27. https://doi.org/10.1096/fj.08-109033.
Sims, G. P., Rowe, D. C., Rietdijk, S. T., Herbst, R., and Coyle, A. J. (2010) HMGB1 and RAGE in inflammation and cancer. Annu Rev Immunol 28, 367-388
Sparvero, Louis J., Denise Asafu-Adjei, Rui Kang, Daolin Tang, Neilay Amin, Jaehyun Im, Ronnye Rutledge, et al. «RAGE (Receptor for Advanced Glycation Endproducts), RAGE Ligands, and Their Role in Cancer and Inflammation». Journal of Translational Medicine 7 (17 Mar. 2009): 17. https://doi.org/10.1186/1479-5876-7-17.
Yonekura, Hideto, Yasuhiko Yamamoto, Shigeru Sakurai, Ralica G. Petrova, Md Joynal Abedin, Hui Li, Kiyoshi Yasui, et al. «Novel Splice Variants of the Receptor for Advanced Glycation End-Products Expressed in Human Vascular Endothelial Cells and Pericytes, and Their Putative Roles in Diabetes-Induced Vascular Injury». The Biochemical Journal 370, no Pt 3 (15 Mar. 2003): 1097-1109. https://doi.org/10.1042/BJ20021371.

1-16. (canceled)

17. A VHH molecule of formula FR1-CDR1-FR2-CDR2-FR3-CDR3-FR4, wherein said VHH molecule binds a Receptor for Advanced Glycation Endproducts (RAGE) at the surface of a lung cell, wherein said VHH molecule comprises:
a CDR1 sequence selected from SEQ ID NOs: 1, 5, 9, 13 or 17, or a variant thereof having at least 60% amino acid identity to any one of said sequences over the entire length thereof,
a CDR2 sequence selected from SEQ ID NOs: 2, 6, 10, 14 or 18, or a variant thereof having at least 60% amino acid identity to any one of said sequences over the entire length thereof, and/or
a CDR3 sequence selected from SEQ ID NOs: 3, 7, 11, 15 or 19, or a variant thereof having at least 60% amino acid identity to any one of said sequences over the entire length thereof.

18. The VHH molecule of claim 17, wherein said VHH molecule comprises SEQ ID NOs: 1, 2 and 3; or SEQ ID NOs: 5, 6 and 7; or SEQ ID NOs: 9, 10 and 11; or SEQ ID NOs: 13, 14 and 15; or SEQ ID NOs: 17, 18 and 19.

19. The VHH molecule of claim 17, wherein said VHH molecule comprises an amino acid sequence selected from any one of SEQ ID NOs: 4, 8, 12, 16 and 20.

20. The VHH molecule of claim 17, wherein the VHH molecule further comprises a tag and/or a linker.

21. The VHH molecule of claim 17, wherein said VHH molecule is humanized.

22. The VHH molecule of claim 17, wherein said VHH molecule binds RAGE with an affinity (Kd) of 0.1 nM to 10 μM.

23. A nucleic acid encoding a VHH molecule of claim 17.

24. A vector comprising a nucleic acid of claim 23, optionally operably linked to a promoter.

25. A recombinant host cell containing a nucleic acid of claim 23, or a vector comprising said nucleic acid.

26. A conjugate compound comprising one or more VHH molecules of claim 17 conjugated to at least one additional compound.

27. The conjugate compound of claim 26, wherein the at least one additional compound is a stabilizing group which is selected from an antibody or a fragment thereof, a Fc fragment, a VHH molecule, PEG, a serum albumin protein or a serum albumin-binding moiety.

28. The conjugate compound of claim 26, wherein the at least one additional compound is a therapeutic, diagnostic or imaging compound, or a vehicle comprising said therapeutic, diagnostic or imaging compound.

29. The conjugate compound of claim 28, wherein the therapeutic compound is selected from a peptide, a polypeptide, a protein, an antibody or a nucleic acid.

30. The conjugate compound of claim 28, wherein the vehicle is selected from a virus, a virus-like particle (VLP), a Cell-Derived Vesicle (CDV), an exosome, a lipid vehicle, a polymer vehicle, a lipid nanoparticle (LNP), a micelle or a liposome.

31. The conjugate compound of claim 26, wherein the conjugate comprises i) one or more VHH molecules, ii) a stabilizing group, iii) a therapeutic, diagnostic or imaging compound, and optionally iv) a vehicle, in any order.

32. A pharmaceutical composition comprising the conjugate compound of claim 26 and a pharmaceutically acceptable support.


SEQUENCE LISTING (129 sequences)

SEQ ID NO: 1 - protein, 8 aa, synthetic construct
GSIFSSAT

SEQ ID NO: 2 - protein, 7 aa, synthetic construct
ISSGGST

SEQ ID NO: 3 - protein, 11 aa, synthetic construct
RLQANFGLVDY

SEQ ID NO: 4 - protein, 117 aa, synthetic construct
QVQLVQSGGGLVQPGGSLRLSCAVSGSIFSSATMGWYRQAPGKQRELAARISSGGSTNYLDSVKGRFTISRDNAKNTVYLQMNSLKLEDTAVYYCRLQANFGLVDYWGKGTQVTVSS

SEQ ID NO: 5 - protein, 8 aa, synthetic construct
GSVSSIYA

SEQ ID NO: 6 - protein, 7 aa, synthetic construct
ITGRGIT

SEQ ID NO: 7 - protein, 10 aa, synthetic construct
KIDRRGYAPY

SEQ ID NO: 8 - protein, 116 aa, synthetic construct
QVQLVQSGGGLVQAGGSLRLSCAASGSVSSIYAMGWYRQAPGKQREWVTTITGRGITDYAASVKGRFTISRDTAKNAVYLQMNNLKPEDTARYYCKIDRRGYAPYWGQGTQVTVSS

SEQ ID NO: 9 - protein, 8 aa, synthetic construct
GRTFSNYA

SEQ ID NO: 10 - protein, 8 aa, synthetic construct
ISWSGGRT

SEQ ID NO: 11 - protein, 19 aa, synthetic construct
ATEAGAFGGVYSSTGSYDY

SEQ ID NO: 12 - protein, 126 aa, synthetic construct
QVQLVQSGGGLVQAGGSLRLSCVASGRTFSNYAMAWFRQAPGEEREFVARISWSGGRTSYADSVKGRFTISRDNAKNTVYLQMISLKPEDTAVYYCATEAGAFGGVYSSTGSYDYWGQGTQVTVSS

SEQ ID NO: 13 - protein, 8 aa, synthetic construct
GRTFSNYA

SEQ ID NO: 14 - protein, 8 aa, synthetic construct
ISWRGGRT

SEQ ID NO: 15 - protein, 19 aa, synthetic construct
ASEGGAFGGVYSATGSYDY

SEQ ID NO: 16 - protein, 126 aa, synthetic construct
EVQLVESGGGLVQAGGSLRLSCVASGRTFSNYAMAWFRQAPGEEREFVARISWRGGRTSYADSVKGRFTISRDNAKNTVYLQMISLKPEDTAVYYCASEGGAFGGVYSATGSYDYWGQGTQVTVSS

SEQ ID NO: 17 - protein, 8 aa, synthetic construct
GTIFSLNP

SEQ ID NO: 18 - protein, 7 aa, synthetic construct
ISDSGTA

SEQ ID NO: 19 - protein, 10 aa, synthetic construct
RSTFAAGNFY

SEQ ID NO: 20 - protein, 116 aa, synthetic construct
EVQLVESGGGLVQPGGSLRLSCAASGTIFSLNPMGWYRQAPGKQLELVALISDSGTANYADFVRGRFTISRDTTKKTLYLQMNSLEPEDTAVYYCRSTFAAGNFYWGQGTQVTVSS

SEQ ID NO: 21 - DNA, 351 bp, synthetic construct
  source 1..351: mol_type = other DNA
caggtgcagctggtgcagtctgggggaggcttggtgcagcctggggggtctctgagactctcctgtgcagtctctggaagtatcttcagtagtgctaccatgggctggtaccgccaggctccggggaagcagcgcgagttggccgcacgtatttctagtggtggtagcacaaactatttagactccgtgaagggccgattcaccatctccagagacaacgccaagaacacggtgtatctacaaatgaacagtctgaaacttgaggacacggccgtctattactgtaggttgcaggctaattttggcctcgtggactattggggcaaagggacccaggtcaccgtctcctca

SEQ ID NO: 22 - DNA, 429 bp, synthetic construct
  source 1..429: mol_type = other DNA
caggtgcagctggtgcagtctgggggaggcttggtgcagcctggggggtctctgagactctcctgtgcagtctctggaagtatcttcagtagtgctaccatgggctggtaccgccaggctccggggaagcagcgcgagttggccgcacgtatttctagtggtggtagcacaaactatttagactccgtgaagggccgattcaccatctccagagacaacgccaagaacacggtgtatctacaaatgaacagtctgaaacttgaggacacggccgtctattactgtaggttgcaggctaattttggcctcgtggactattggggcaaagggacccaggtcaccgtctcctcagcggccgcagaacaaaaactcatctcagaagaggatctgaatggggccgcacatcaccaccatcaccatgggagctag

SEQ ID NO: 23 - DNA, 348 bp, synthetic construct
  source 1..348: mol_type = other DNA
caggtgcagctggtgcagtctgggggaggcttggtgcaggctggggggtctctgagactctcctgtgcagcctctggaagcgtctccagtatttatgccatgggctggtaccggcaggctcccgggaagcagcgcgagtgggtcacaactatcactggtcgtggtattaccgactatgcagcctccgtgaagggccggttcaccatctccagagacaccgccaagaacgcggtatatctccaaatgaacaacctgaaacctgaggacacggcccgctattactgcaagatagatcgtaggggctatgcgccctactggggccaggggacccaggtcactgtctcctca

SEQ ID NO: 24 - DNA, 426 bp, synthetic construct
  source 1..426: mol_type = other DNA
caggtgcagctggtgcagtctgggggaggcttggtgcaggctggggggtctctgagactctcctgtgcagcctctggaagcgtctccagtatttatgccatgggctggtaccggcaggctcccgggaagcagcgcgagtgggtcacaactatcactggtcgtggtattaccgactatgcagcctccgtgaagggccggttcaccatctccagagacaccgccaagaacgcggtatatctccaaatgaacaacctgaaacctgaggacacggcccgctattactgcaagatagatcgtaggggctatgcgccctactggggccaggggacccaggtcactgtctcctcagcggccgcagaacaaaaactcatctcagaagaggatctgaatggggccgcacatcaccaccatcaccatgggagctag

SEQ ID NO: 25 - DNA, 377 bp, synthetic construct
  source 1..377: mol_type = other DNA
aggtgcagctggtgcagtctgggggaggattggtgcaggctgggggctctctgagactctcctgtgtagcctctggacgcaccttcagtaactatgccatggcctggttccgccaggctccaggcgaggagcgtgagtttgtagcacgtattagttggagcggtggtaggacatcatatgcagactccgtgaagggccgattcaccatctccagagacaacgccaagaacacggtgtatctgcaaatgatcagcctgaaacctgaggacacggccgtttattactgtgcaacagaggccggcgcttttggtggtgtatactcctctacagggtcttatgactactggggccaggggacccaggtcactgtctcctca

SEQ ID NO: 26 - DNA, 455 bp, synthetic construct
  source 1..455: mol_type = other DNA
aggtgcagctggtgcagtctgggggaggattggtgcaggctgggggctctctgagactctcctgtgtagcctctggacgcaccttcagtaactatgccatggcctggttccgccaggctccaggcgaggagcgtgagtttgtagcacgtattagttggagcggtggtaggacatcatatgcagactccgtgaagggccgattcaccatctccagagacaacgccaagaacacggtgtatctgcaaatgatcagcctgaaacctgaggacacggccgtttattactgtgcaacagaggccggcgcttttggtggtgtatactcctctacagggtcttatgactactggggccaggggacccaggtcactgtctcctcagcggccgcagaacaaaaactcatctcagaagaggatctgaatggggccgcacatcaccaccatcaccatgggagctag

SEQ ID NO: 27 - DNA, 378 bp, synthetic construct
  source 1..378: mol_type = other DNA
gaggtgcagctggtggagtctgggggaggattggtgcaggctgggggctctctgagactctcctgtgtagcctctggacgcaccttcagtaactatgccatggcctggttccgccaggctccaggcgaggagcgtgagtttgtagcacgtattagttggcgcggtggtaggacatcatatgcagactccgtgaagggccgattcaccatctccagagacaacgccaagaacacggtgtatctgcaaatgatcagcctgaaacctgaggacacggccgtttattactgtgcatcagagggcggcgcttttggtggtgtgtactccgctacagggtcttatgactactggggccaggggacccaggtcactgtctcctca

SEQ ID NO: 28 - DNA, 456 bp, synthetic construct
  source 1..456: mol_type = other DNA
gaggtgcagctggtggagtctgggggaggattggtgcaggctgggggctctctgagactctcctgtgtagcctctggacgcaccttcagtaactatgccatggcctggttccgccaggctccaggcgaggagcgtgagtttgtagcacgtattagttggcgcggtggtaggacatcatatgcagactccgtgaagggccgattcaccatctccagagacaacgccaagaacacggtgtatctgcaaatgatcagcctgaaacctgaggacacggccgtttattactgtgcatcagagggcggcgcttttggtggtgtgtactccgctacagggtcttatgactactggggccaggggacccaggtcactgtctcctcagcggccgcagaacaaaaactcatctcagaagaggatctgaatggggccgcacatcaccaccatcaccatgggagctag

SEQ ID NO: 29 - DNA, 348 bp, synthetic construct
  source 1..348: mol_type = other DNA
gaggtgcagctggtggagtctgggggaggcttggtgcagcctggggggtctctgagactctcctgtgcagcctctggaaccatcttcagtctcaatcccatgggctggtaccgccaggctccagggaaacagctcgagttggtcgcactaatttcggatagtggaaccgcaaactatgcagacttcgtgaggggccgattcaccatctccagagacaccaccaagaaaacgctatatctgcagatgaacagccttgaacctgaggacacagccgtctattactgtagatctacattcgcggcggggaatttttactggggccaggggacccaggtcactgtctcctca

SEQ ID NO: 30 - DNA, 426 bp, synthetic construct
  source 1..426: mol_type = other DNA
gaggtgcagctggtggagtctgggggaggcttggtgcagcctggggggtctctgagactctcctgtgcagcctctggaaccatcttcagtctcaatcccatgggctggtaccgccaggctccagggaaacagctcgagttggtcgcactaatttcggatagtggaaccgcaaactatgcagacttcgtgaggggccgattcaccatctccagagacaccaccaagaaaacgctatatctgcagatgaacagccttgaacctgaggacacagccgtctattactgtagatctacattcgcggcggggaatttttactggggccaggggacccaggtcactgtctcctcagcggccgcagaacaaaaactcatctcagaagaggatctgaatggggccgcacatcaccaccatcaccatgggagctag

SEQ ID NO: 31 - protein, 142 aa, synthetic construct
QVQLVQSGGGLVQPGGSLRLSCAVSGSIFSSATMGWYRQAPGKQRELAARISSGGSTNYLDSVKGRFTISRDNAKNTVYLQMNSLKLEDTAVYYCRLQANFGLVDYWGKGTQVTVSSAAAEQKLISEEDLNGAAHHHHHHGS

SEQ ID NO: 32 - protein, 141 aa, synthetic construct
QVQLVQSGGGLVQAGGSLRLSCAASGSVSSIYAMGWYRQAPGKQREWVTTITGRGITDYAASVKGRFTISRDTAKNAVYLQMNNLKPEDTARYYCKIDRRGYAPYWGQGTQVTVSSAAAEQKLISEEDLNGAAHHHHHHGS

SEQ ID NO: 33 - protein, 151 aa, synthetic construct
QVQLVQSGGGLVQAGGSLRLSCVASGRTFSNYAMAWFRQAPGEEREFVARISWSGGRTSYADSVKGRFTISRDNAKNTVYLQMISLKPEDTAVYYCATEAGAFGGVYSSTGSYDYWGQGTQVTVSSAAAEQKLISEEDLNGAAHHHHHHGS

SEQ ID NO: 34 - protein, 151 aa, synthetic construct
EVQLVESGGGLVQAGGSLRLSCVASGRTFSNYAMAWFRQAPGEEREFVARISWRGGRTSYADSVKGRFTISRDNAKNTVYLQMISLKPEDTAVYYCASEGGAFGGVYSATGSYDYWGQGTQVTVSSAAAEQKLISEEDLNGAAHHHHHHGS

SEQ ID NO: 35 - protein, 141 aa, synthetic construct
EVQLVESGGGLVQPGGSLRLSCAASGTIFSLNPMGWYRQAPGKQLELVALISDSGTANYADFVRGRFTISRDTTKKTLYLQMNSLEPEDTAVYYCRSTFAAGNFYWGQGTQVTVSSAAAEQKLISEEDLNGAAHHHHHHGS

SEQ ID NO: 36 - protein, 25 aa, synthetic construct
AAAEQKLISEEDLNGAAHHHHHHGS

SEQ ID NO: 37 - protein, 6 aa, synthetic construct
HHHHHH

SEQ ID NO: 38 - protein, 10 aa, synthetic construct
EQKLISEEDL

SEQ ID NO: 39 - protein, 4 aa, synthetic construct
GGGG

SEQ ID NO: 40 - protein, 5 aa, synthetic construct
GGGGG

SEQ ID NO: 41 - protein, 6 aa, synthetic construct
GGGGGG

SEQ ID NO: 42 - protein, 6 aa, synthetic construct
GGGLQR

SEQ ID NO: 43 - protein, 7 aa, synthetic construct
GGGGLQR

SEQ ID NO: 44 - protein, 8 aa, synthetic construct
GGGGGLQR

SEQ ID NO: 45 - protein, 9 aa, synthetic construct
GGGGGGLQR

SEQ ID NO: 46 - protein, 10 aa, synthetic construct
GGGGGGGLQR

SEQ ID NO: 47 - protein, 15 aa, synthetic construct
AAAHHHHHHGGGLQR

SEQ ID NO: 48 - protein, 16 aa, synthetic construct
AAAHHHHHHGGGGLQR

SEQ ID NO: 49 - protein, 17 aa, synthetic construct
AAAHHHHHHGGGGGLQR

SEQ ID NO: 50 - protein, 18 aa, synthetic construct
AAAHHHHHHGGGGGGLQR

SEQ ID NO: 51 - protein, 19 aa, synthetic construct
AAAHHHHHHGGGGGGGLQR

SEQ ID NO: 52 - DNA, 24 bp, synthetic construct
  source 1..24: mol_type = other DNA
ggaagtatcttcagtagtgctacc

SEQ ID NO: 53 - DNA, 24 bp, synthetic construct
  source 1..24: mol_type = other DNA
ggaagcgtctccagtatttatgcc

SEQ ID NO: 54 - DNA, 24 bp, synthetic construct
  source 1..24: mol_type = other DNA
ggacgcaccttcagtaactatgcc

SEQ ID NO: 55 - DNA, 24 bp, synthetic construct
  source 1..24: mol_type = other DNA
ggacgcaccttcagtaactatgcc

SEQ ID NO: 56 - DNA, 24 bp, synthetic construct
  source 1..24: mol_type = other DNA
ggaaccatcttcagtctcaatccc

SEQ ID NO: 57 - DNA, 21 bp, synthetic construct
  source 1..21: mol_type = other DNA
atttctagtggtggtagcaca

SEQ ID NO: 58 - DNA, 21 bp, synthetic construct
  source 1..21: mol_type = other DNA
atcactggtcgtggtattacc

SEQ ID NO: 59 - DNA, 24 bp, synthetic construct
  source 1..24: mol_type = other DNA
attagttggagcggtggtaggaca

SEQ ID NO: 60 - DNA, 24 bp, synthetic construct
  source 1..24: mol_type = other DNA
attagttggcgcggtggtaggaca

SEQ ID NO: 61 - DNA, 21 bp, synthetic construct
  source 1..21: mol_type = other DNA
atttcggatagtggaaccgca

SEQ ID NO: 62 - DNA, 33 bp, synthetic construct
  source 1..33: mol_type = other DNA
aggttgcaggctaattttggcctcgtggactat

SEQ ID NO: 63 - DNA, 30 bp, synthetic construct
  source 1..30: mol_type = other DNA
aagatagatcgtaggggctatgcgccctac

SEQ ID NO: 64 - DNA, 57 bp, synthetic construct
  source 1..57: mol_type = other DNA
gcaacagaggccggcgcttttggtggtgtatactcctctacagggtcttatgactac

SEQ ID NO: 65 - DNA, 57 bp, synthetic construct
  source 1..57: mol_type = other DNA
gcatcagagggcggcgcttttggtggtgtgtactccgctacagggtcttatgactac

SEQ ID NO: 66 - DNA, 30 bp, synthetic construct
  source 1..30: mol_type = other DNA
agatctacattcgcggcggggaatttttac

SEQ ID NO: 67 - DNA, 78 bp, synthetic construct
  source 1..78: mol_type = other DNA
gcggccgcagaacaaaaactcatctcagaagaggatctgaatggggccgcacatcaccaccatcaccatgggagctag

SEQ ID NO: 68 - DNA, 30 bp, synthetic construct
  source 1..30: mol_type = other DNA
atatatgagctctggaaggaagcaggatgg

SEQ ID NO: 69 - DNA, 34 bp, synthetic construct
  source 1..34: mol_type = other DNA
ttaattccgcggaggccctccagtactactctcg

SEQ ID NO: 70 - DNA, 40 bp, synthetic construct
  source 1..40: mol_type = other DNA
gggatccaccggtcgccaccatgccagcggggacagcagc

SEQ ID NO: 71 - DNA, 41 bp, synthetic construct
  source 1..41: mol_type = other DNA
tcctcgcccttgctcaccatcggtcccccggcaccattctc

SEQ ID NO: 72 - DNA, 1215 bp, Homo sapiens
  source 1..1215: mol_type = genomic DNA
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttga

SEQ ID NO: 73 - DNA, 1209 bp, Mus musculus
  source 1..1209: mol_type = genomic DNA
atgccagcggggacagcagctagagcctgggtgctggttcttgctctatggggagctgtagctggtggtcagaacatcacagcccggattggagagccacttgtgctaagctgtaagggggcccctaagaagccgccccagcagctagaatggaaactgaacacaggaagaactgaagcttggaaggtcctctctccccagggaggcccctgggacagcgtggctcgaatcctccccaatggttccctcctccttccagccactggaattgtcgatgaggggactttccggtgtcgggcaactaacaggcgagggaaggaggtcaagtccaactaccgagtccgagtctaccagattcctgggaagccagaaattgtggatcctgcctctgaactcacagccagtgtccctaataaggtggggacatgtgtgtctgagggaagctaccctgcagggacccttagctggcacttagatgggaaacttctgattcccgatggcaaagaaacactcgtgaaggaagagaccaggagacaccctgagacgggactctttacactgcggtcagagctgacagtgatccccacccaaggaggaacccatcctaccttctcctgcagtttcagcctgggccttccccggcgcagacccctgaacacagcccccatccaactccgagtcagggagcctgggcctccagagggcattcagctgttggttgagcctgaaggtggaatagtcgctcctggtgggactgtgaccttgacctgtgccatctctgcccagccccctcctcaggtccactggataaaggatggtgcacccttgcccctggctcccagccctgtgctgctcctccctgaggtggggcacgaggatgagggcacctatagctgcgtggccacccaccctagccacggacctcaggaaagccctcctgtcagcatcagggtcacagaaaccggcgatgaggggccagctgaaggctctgtgggtgagtctgggctgggtacgctagccctggccttggggatcctgggaggcctgggagtagtagccctgctcgtcggggctatcctgtggcgaaaacgacaacccaggcgtgaggagaggaaggccccggaaagccaggaggatgaggaggaacgtgcagagctgaatcagtcagaggaagcggagatgccagagaatggtgccgggggaccgtaa

SEQ ID NO: 74 - protein, 12 aa, synthetic construct
GGGGSCHHHHHH

SEQ ID NO: 75 - protein, 25 aa, synthetic construct [sequence omitted]

SEQ ID NO: 76 - protein, 25 aa, synthetic construct
QVQLVQSGGGLVQPGGSLRLSCAVS

SEQ ID NO: 77 - protein, 25 aa, synthetic construct
QVQLVQSGGGLVQAGGSLRLSCAAS

SEQ ID NO: 78 - protein, 25 aa, synthetic construct
QVQLVQSGGGLVQAGGSLRLSCVAS

SEQ ID NO: 79 - protein, 25 aa, synthetic construct
EVQLVESGGGLVQAGGSLRLSCVAS

SEQ ID NO: 80 - protein, 25 aa, synthetic construct [sequence omitted]

SEQ ID NO: 81 - protein, 25 aa, synthetic construct
QVQLVESGGGLVQPGGSLRLSCAAS

SEQ ID NO: 82 - protein, 25 aa, synthetic construct
EVQLQESGGGLVQPGGSLRLSCAAS

SEQ ID NO: 83 - protein, 25 aa, synthetic construct
EVQLVQSGGGLVQPGGSLRLSCAAS

SEQ ID NO: 84 - protein, 25 aa, synthetic construct
EVQLVESGGGLVQAGGSLRLSCAAS

SEQ ID NO: 85 - protein, 25 aa, synthetic construct
EVQLVESGGGLVQPGGSLRLSCVAS

SEQ ID NO: 86 - protein, 25 aa, synthetic construct [sequence omitted]

SEQ ID NO: 87 - protein, 17 aa, synthetic construct
MGWYRQAPGKQRELVAR

SEQ ID NO: 88 - protein, 17 aa, synthetic construct
MGWYRQAPGKQRELAAR

SEQ ID NO: 89 - protein, 17 aa, synthetic construct
MGWYRQAPGKQREWVTT

SEQ ID NO: 90 - protein, 17 aa, synthetic construct
MAWFRQAPGEEREFVAR

SEQ ID NO: 91 - protein, 17 aa, synthetic construct
MGWYRQAPGKQLELVAL

SEQ ID NO: 92 - protein, 17 aa, synthetic construct
MAWYRQAPGKQRELVAR

SEQ ID NO: 93 - protein, 17 aa, synthetic construct
MGWFRQAPGKQRELVAR

SEQ ID NO: 94 - protein, 17 aa, synthetic construct
MGWYRQAPGEQRELVAR

SEQ ID NO: 95 - protein, 17 aa, synthetic construct
MGWYRQAPGKERELVAR

SEQ ID NO: 96 - protein, 17 aa, synthetic construct
MGWYRQAPGKQLELVAR

SEQ ID NO: 97 - protein, 17 aa, synthetic construct
MGWYRQAPGKQREFVAR

SEQ ID NO: 98 - protein, 17 aa, synthetic construct
MGWYRQAPGKQREWVAR

SEQ ID NO: 99 - protein, 17 aa, synthetic construct
MGWYRQAPGKQRELVTR

SEQ ID NO: 100 - protein, 17 aa, synthetic construct
MGWYRQAPGKQRELVAT

SEQ ID NO: 101 - protein, 17 aa, synthetic construct
MGWYRQAPGKQRELVAL

SEQ ID NO: 102 - protein, 38 aa, synthetic construct
NYADSVKGRFTISRDNAKNTVYLQMNSLKPEDTAVYYC

SEQ ID NO: 103 - protein, 38 aa, synthetic construct
NYLDSVKGRFTISRDNAKNTVYLQMNSLKLEDTAVYYC

SEQ ID NO: 104 - protein, 38 aa, synthetic construct
DYAASVKGRFTISRDTAKNAVYLQMNNLKPEDTARYYC

SEQ ID NO: 105 - protein, 38 aa, synthetic construct
SYADSVKGRFTISRDNAKNTVYLQMISLKPEDTAVYYC

SEQ ID NO: 106 - protein, 38 aa, synthetic construct
NYADFVRGRFTISRDTTKKTLYLQMNSLEPEDTAVYYC

SEQ ID NO: 107 - protein, 38 aa, synthetic construct
SYADSVKGRFTISRDNAKNTVYLQMNSLKPEDTAVYYC

SEQ ID NO: 108 - protein, 38 aa, synthetic construct
DYADSVKGRFTISRDNAKNTVYLQMNSLKPEDTAVYYC

SEQ ID NO: 109 - protein, 38 aa, synthetic construct
NYLDSVKGRFTISRDNAKNTVYLQMNSLKPEDTAVYYC

SEQ ID NO: 110 - protein, 38 aa, synthetic construct
NYAASVKGRFTISRDNAKNTVYLQMNSLKPEDTAVYYC

SEQ ID NO: 111 - protein, 38 aa, synthetic construct
NYADFVKGRFTISRDNAKNTVYLQMNSLKPEDTAVYYC

SEQ ID NO: 112 - protein, 38 aa, synthetic construct
NYADSVRGRFTISRDNAKNTVYLQMNSLKPEDTAVYYC

SEQ ID NO: 113 - protein, 38 aa, synthetic construct
NYADSVKGRFTISRDTAKNTVYLQMNSLKPEDTAVYYC

SEQ ID NO: 114 - protein, 38 aa, synthetic construct
NYADSVKGRFTISRDNTKNTVYLQMNSLKPEDTAVYYC

SEQ ID NO: 115 - protein, 38 aa, synthetic construct
NYADSVKGRFTISRDNAKKTVYLQMNSLKPEDTAVYYC

SEQ ID NO: 116 - protein, 38 aa, synthetic construct
NYADSVKGRFTISRDNAKNAVYLQMNSLKPEDTAVYYC

SEQ ID NO: 117 - protein, 38 aa, synthetic construct
NYADSVKGRFTISRDNAKNTLYLQMNSLKPEDTAVYYC

SEQ ID NO: 118 - protein, 38 aa, synthetic construct
NYADSVKGRFTISRDNAKNTVYLQMISLKPEDTAVYYC

SEQ ID NO: 119 - protein, 38 aa, synthetic construct
NYADSVKGRFTISRDNAKNTVYLQMNNLKPEDTAVYYC

SEQ ID NO: 120 - protein, 38 aa, synthetic construct
NYADSVKGRFTISRDNAKNTVYLQMNSLEPEDTAVYYC

SEQ ID NO: 121 - protein, 38 aa, synthetic construct
NYADSVKGRFTISRDNAKNTVYLQMNSLKLEDTAVYYC

SEQ ID NO: 122 - protein, 38 aa, synthetic construct
NYADSVKGRFTISRDNAKNTVYLQMNSLKPEDTARYYC

SEQ ID NO: 123 - protein, 11 aa, synthetic construct
WGQGTQVTVSS

SEQ ID NO: 124 - protein, 11 aa, synthetic construct
WGKGTQVTVSS

SEQ ID NO: 125 - protein, 11 aa, synthetic construct
WGQGTQVTVSS

SEQ ID NO: 126 - protein, 17 aa, synthetic construct
MGWYRQAPGKGLELVAR

SEQ ID NO: 127 - protein, 4 aa, synthetic construct
HHHH

SEQ ID NO: 128 - protein, 6 aa, synthetic construct
HHHHHH

SEQ ID NO: 129 - protein, 8 aa, synthetic construct
HHHHHHHH